(19) United States
(12) Patent Application Publication
(10) Pub. No.: US 2026/0083698 A1
(43) Pub. Date: Mar. 26, 2026

(54) Synergistic Stimulation of Mucociliary Clearance to Treat Mucus Obstruction in Cystic Fibrosis and Other Muco-Obstructive Disorders

(71) Applicant: The Board of Trustees of the Leland Stanford Junior University, Stanford, CA (US)

(72) Inventors: Jeffrey J. Wine, Stanford, CA (US); Nam Soo Joo, Stanford, CA (US); Carlos E. Milla, Stanford, CA (US)

(21) Appl. No.: 19/110,980

(22) PCT Filed: Sep. 19, 2023
(86) PCT No.: PCT/US2023/033160

§ 371 Date: Mar. 12, 2025

Related U.S. Application Data
(60) Provisional application No. 63/408,596, filed on Sep. 21, 2022.

(51) Int. Cl. A61K 31/352; A61K 31/27; A61P 11/12

(52) U.S. Cl. CPC A61K 31/352; A61K 31/27; A61P 11/12

(57) ABSTRACT

The present disclosure provides methods of treating an individual for a muco-obstructive, the methods including: administering to the individual a b-adrenergic agonist or an adenylate cyclase activator, in combination with a cholinergic agonist to treat the individual for the muco-obstructive disorder.

### CROSS-REFERENCE TO RELATED APPLICATION

This application claims benefit of U.S. Provisional Patent Application No. 63/408,596 , filed Sep. 21, 2022, which application is incorporated herein by reference in its entirety.

### INTRODUCTION

Cystic fibrosis (CF) is a multi-organ syndrome of which the most critical clinical phenotype is airway mucus obstruction, chronic lung infections, and neutrophilic inflammation. Unless arrested, the resulting tissue damage produces a life-long decline in pulmonary function. CF is caused by loss of function mutations in the gene for an anion channel, CFTR, cystic fibrosis transmembrane conductance regulator, which is important for fluid secretion in the airways. CF airways appear normal at birth, but their airway surface liquid (ASL) is less able to kill bacteria (1) and mucus clearance rates are slowed (2). Chronic lung infections are the main drivers of declining lung function in humans (3,4). However even when chronic infection is prevented in transgenic ferrets with CF (“CF ferrets”)with antibiotics, a muco-obstructive phenotype with bronchial obstruction and inflammation persists (5).

Improving mucociliary clearance (MCC) in the airway is then an important therapeutic goal in CF. Improvements in mucus clearance, sometimes sufficient to show clinical efficacy, have been obtained with inhalation therapies with recombinant human DNase (Pulmozyme)(6,7), hypertonic saline (8,9), or powdered mannitol (10). For most people with CF, the most effective improvements in mucus clearance are provided by small molecule modulators that partially restore function in CFTR with specific mutations(11,12). For those patients whose mutations are not treatable with current modulators, or whose lung function declines despite modulators, additional improvements in mucus clearance could be therapeutic. MCC is a function of the volume and composition of the airway surface liquid (ASL), rheological properties of the secreted mucus and ciliary beat frequency (CBF)(13).

### SUMMARY

Provided herein are methods and compositions for the treatment of cystic fibrosis in individuals in need thereof.

The present disclosure provides methods of treating an individual suffering from a muco-obstructive disorder, the methods including: administering to the individual a β-adrenergic agonist or an adenylate cyclase activator, in combination with a cholinergic agonist to treat the individual for the muco-obstructive disorder.

Also provided are methods of increasing the rate of airway submucosal gland secretion in an individual, the methods including: administering to the individual a β-adrenergic agonist or an adenylate cyclase activator, in combination with a cholinergic agonist to increase the rate of submucosal gland secretion in the individual.

The present disclosure also provides methods of suppressing cholinergic agonist induced airway smooth muscle contractions in an individual, the methods including: administering to the individual a β-adrenergic agonist or an adenylyl cyclase activator, wherein the administration of the β-adrenergic agonist of the adenylyl cyclase activator occurs before or at the same time that the individual has been administered a cholinergic agonist to suppress the airway smooth muscle contraction.

Compositions and devices for practicing the subject methods are also provided.

### BRIEF DESCRIPTION OF THE FIGURES

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIG. 1. Synergistic mucus clearance in CF ferret and WT pig tracheas. (A) Time courses of MCC Velocity (MCCV, a measurement of the velocity of MCC) from CF ferrets in response to the direct adenylate cyclase activator 10 μM forskolin (blue open circles, Fsk, n=4), the cholinergic agonist 0.3 μM carbachol (red open squares, Carb, n=4) or the combination (filled symbols). CF ferret genotypes were 5 CFTRG551D, one CFTRΔF/ΔF, and one CFTRG551D/KO. (B) Summary data as box and whisker plots. Bs: basal/unstimulated MCCV, Sum: arithmetic sum of MCCV measurements for the agonists used separately, SR: synergy response, MCCV measured for the combined agonists. SR was 5.7 times larger than sum, representing synergy (P=0.006, n=3-7) (C) MCCV from 2 to 5-day old piglets: same protocol and symbols as for CF ferrets. (D) Summary data. SR was 3.9 times larger than sum, (P=3.8E−05, n=4-8). (E) Responses in pigs to the β-adrenergic agonist 10 μM formoterol/Fmt (blue circles) instead of forskolin, otherwise same protocol and symbols. (F) Summary data. SR was 3.4 times larger than sum, (P=0.005, n=3-7).

FIG. 2. Elevating cAMP by adenylate cyclase activation inhibited muscle tension and airway narrowing to carbachol. (A, B) To measure muscle tension, one end of an isolated WT ferret trachealis muscle bundle was secured in a Sylgard-lined Petri dish filled with KRB solution and the other end was attached by 26-gauge wire to a previously calibrated strain gauge. Tension responses to increasing carbachol doses in the absence (A) and presence (B) of 10 μM forskolin are displayed. (CF) To measure airway narrowing, thin slices (˜2 mm) of tracheal rings from WT pigs and WT or CF ferrets were treated with carbachol alone or in the presence of forskolin (F or Fsk) or formoterol (Fmt); their lumens were imaged over time and the areas measured as an assay for muscle constriction. After a baseline period (black open squares), carbachol was added (0.3 μM, red open squares), or in the presence of 10 μM forskolin or formoterol. (red closed circles). Averaged responses at 10 min intervals are shown for: (C) WT piglet tracheas (n=3-7) with forskolin. (D) WT piglet tracheas with formoterol (n=5). (E) WT ferret tracheas (2 tracheas, 5 experiments). (F). CF ferrets (n=2, one CFTRΔF/ΔF and one CFTRΔF/G551D).

FIG. 3. Combined agonists increase gland mucus secretion synergistically. Average secretion rates are summarized as box and whisker plots for 15 WT pigs (A), 12 WT ferrets (D) and 2 CF ferrets (G) using the same labeling as in FIG. 1. Each agonist increased secretion over baseline values, and rates to the combined agonists (SR, synergy response) were significantly larger than the arithmetical sum (Sum) of their individual responses. (B) Average secretion rates for individual WT pigs to 10 μM forskolin alone and combined with 0.3 μM carbachol (60 glands, 8 pigs). (C) As in B but with carbachol alone and combined with forskolin (60-61 glands, 7 pigs). (E, F) WT adult ferret data with same conditions as in pigs (26-29 glands, 7 ferrets). (H, I) CF ferret data produced with same conditions as pigs and WT ferrets. A single ferret was run in each condition and secretion rates were measured in 7-14 glands. Time courses of average responses are plotted at 10 min intervals.

FIG. 4. Combined agonists inhibit sodium absorption and stimulate anion secretion by surface epithelia. (A) Cartoon of two electrogenic, ion transport pathways across airway apical epithelium: anion secretion increases surface fluid, Na+ absorption decreases surface fluid. The two pathways have opposite effects on fluid depth, but additive effects on short circuit current (Isc) because of their opposite valence and transport directions. (B) Raw trace of Isc across pig tracheal epithelium using Chart 4 software. After reaching a stable, unstimulated Isc (here, >2 h post-mounting), 10 μM forskolin, 0.3 μM carbachol, 10 μM benzamil, 20 μM benzopyrimido-pyrrolo-oxazinedione (BPO-27) and 200 μM niflumic acid were sequentially added at times shown. (C, D) Pig tracheal mucosa: averaged Δ Isc plots over time in response to (C) forskolin alone followed by forskolin+carbachol and (D) reversed order of agonist addition. (E & F) Ferret tracheal mucosa: averaged A Isc plots with same protocol as for pigs. Pig traces (C, D) are based on 10-12 experiments with tissues from 6 to 7 pigs. Ferret traces (E, F) are from 7 experiments with tissues from 5 ferrets.

FIG. 5 Agonists stimulated ciliary beat frequency with additive effects. Ciliary beat frequency was measured in human nasal mucosa from 4 subjects. CBF (in Hz) of unstimulated tissues at 37° C. in Krebs buffer (KRB) was 10.46±0.95. Each agonists alone increased CBF by small amounts that did not reach significance in this small sample: carbachol: 11.04±1.3 (5.3%) and forskolin 12.06±1.22 (9.8%). The combined agonists increased CBF significantly 13.31±0.77 (27.2%, n=4, P<0.05) when comparing difference in CBF (A) to unstimulated CBF (KRB), but not to the arithmetical sum of A CBF to the combined agonists: 2.85±0.76 (synergy paradigm) versus 2.19±0.66 (arithmetic sum) (n=4, P=0.47). (Note that in this experiment any effects on ASL by the agonists were diluted because the ciliated tissue was studied while submerged in Krebs solution.)

FIG. 6 Summary diagram linking increased ASL production to increased mucus clearance. (A) Relationship between Δ Isc, ASL depth and MCCV. MCCV (redrawn from FIG. 1C) is shown on the main graph; inset shows Δ Isc (from FIG. 4C) with time points aligned to the MCCV graph. Dashed brown line is inferred change in ASL depth in the absence of MCC. (B) Cartoons of main electrogenic ion flows across tracheal surface epithelium in four conditions: baseline, β-adrenergic (β-Adn), carbachol (CCh), and β-Adn+CCh. Each panel shows the inferred status of anion secretion, Na+− absorption and resulting changes in ASL depth. The changes of ASL depth are inferred. In our experiments, the main change is an increased velocity of mucus clearance, which will tend to counteract the ASL depth increase. Because carbachol inhibits Na+ (and fluid) absorption and stimulates anion (and fluid) secretion, the combined agonists have opposite effects on Isc, but at least additive increases on ASL depth. (C) Summary diagram of component processes leading to synergistic increases in MCCV in ex vivo tracheas of WT ferrets, WT pigs and CF ferrets. The net result is a marked increase in MCCV.

FIG. 7 Individual MCCV responses for 7 CF ferrets to the synergy paradigm. (A-G) Protocols and genotypes are shown on each time-MCC velocity plot. Note that a reduced y-axis scale was used (E) and (G) to show synergistic MCCV. Also note that the averaged MCCV of T10-30 to 0.3 μM carbachol in (E) is less than 5% of those in (A) and (C).

FIG. 8 depicts synergistic MCC by sequential agonists with methacholine and formoterol.

FIG. 9 depicts the protective effect of formoterol on methacholine induced muscle contraction.

FIG. 10 depicts simultaneous treatment with formoterol and methacholine inducing the synergistic response.

FIG. 11 depicts simultaneous treatment with formoterol and methacholine does not induce muscle contraction.

FIG. 12 depicts mean squared displacement of particles transported with ASL in cystic fibrosis and healthy mature human nasal epithelial cell cultures in response to treatment with either DMSO control, Forskolin, carbachol, forskolin+carbachol (SP) or the CFTR modulators combination Elexacaftor (3 μM)-Tezacaftor (3 μM)-Ivacaftor (10 μM) (ETI).

FIG. 13 depicts effective diffusivity of mucus transport in cystic fibrosis and healthy mature human nasal epithelial cell cultures in response to treatment with either DMSO control, Forskolin, carbachol, forskolin+carbachol (SP) or the combination Elexacaftor (3 μM)-Tezacaftor (3 μM)-Ivacaftor (10 μM) (ETI).

FIG. 14 depicts the effects of formoterol (Fmt) and Fmt+Methacholine (MCh) on TMV in a Sheep cystic fibrosis Model.

FIG. 15 depicts the effects of formoterol (Fmt) and Fmt+Methacholine (MCh) on whole lung clearance in a Sheep CF Model.

FIG. 16 depicts the effects of Albuterol+Methacholine in single ascending dose tolerability in Healthy Volunteers.

FIG. 17 depicts the effects of Formoterol+Methacholine in single ascending dose tolerability in Healthy Volunteers.

FIG. 18 depicts the effects of Formoterol+Methacholine in single ascending dose tolerability in CF patients.

FIG. 19 depicts sputum production (in grams) by the CF patients in the single ascending dose tolerability study.

FIG. 20 depicts the percentage of percent solids content in the sputum produced by the CF patients in the single ascending dose tolerability study.

FIG. 21 ENaC inhibition increases baseline MCCV but is not additive to the dual agonist stimulation. Time courses of MCCV in response to 10 μM benzamil (Bz, red open squares) or dual agonist in the presence (closed squares) and absence (open blue triangles) of benzamil from WT newborn piglet tracheas (n=4, each).

FIG. 22 Increased HCO3-secretion by the synergy agonists. Synergy agonists significantly increased HCO3-secretion rate when compared to baseline conditions (p=2E-05, n=13 from 8 pig tracheas).

FIG. 23 The synergy agonists increase ASL height in ex vivo WT pig tracheas. A. Averaged ASL height changes by DMSO (open black squares, n=2 pigs) or the drugs (filled orange squares, n=4 pigs). B. Summary of rates of ASL height increases in baseline (Bs), formoterol, and synergy agonists.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It must be noted that as used herein and in the appended claims, the singular forms “a”, “an”, and “the” include plural referents unless the context clearly dictates otherwise. For example, the term “a β-adrenergic agonist” refers to one or more β-adrenergic agonists, i.e., a single β-adrenergic agonist and multiple β-adrenergic agonists. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer containing purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a protein, a cell, or an organism, refers to a nucleic acid, protein, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “exogenous” as used herein as applied to a nucleic acid or a protein refers to a nucleic acid or protein that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell. As used herein, the term “endogenous polypeptide” refers to a polypeptide that is normally found in and/or produced by a given bacterium, organism, or cell in nature.

“Recombinant,” as used herein, means that a particular nucleic acid or protein is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA containing the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

Thus, e.g., the term “recombinant” nucleic acid or “recombinant” protein refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The term “biological sample” encompasses a clinical sample or a non-clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample containing polynucleotides and/or polypeptides that is obtained from a patient's sample cell (e.g., a cell lysate or other cell extract containing polynucleotides and/or polypeptides); and a sample containing sample cells from a patient. A biological sample containing a sample cell from a patient can also include normal, non-diseased cells. A biological sample may be from a plant or an animal. The biological sample may also be from any species. In certain embodiments of the method, the biological sample includes a cell. In some instances of the method, the cell is in vitro. In some instances of the method, the cell is in vivo.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies (see, e.g., Hamers-Casterman et al. (1993) Nature 363:446; Desmyter et al. (2015) Curr. Opin. Struct. Biol. 32:1); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

A “single-chain antibody,” “single chain variable fragment,” or “scFv” has an antibody heavy chain variable domain (VH) and a light-chain variable domain (VL) joined together by a flexible peptide linker. The peptide linker is typically 10-25 amino acids in length. Single-chain antibodies retain the antigen-binding properties of natural full-length antibodies, but are smaller than natural intact antibodies or Fab fragments because of the lack of an Fc domain.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” include old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody. Nanobodies are smaller than human antibodies, where nanobodies are generally 12-15 kDa, human antibodies are generally 150-160 kDa, Fab fragments are ˜50 kDa and single-chain variable fragments are ˜25 kDa. Nanobodies provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like. Specific nanobodies have been successfully generated using yeast surface display as shown in McMahon et al. (2018) Nature Structural Molecular Biology 25(3):289-296 which is specifically incorporated herein by reference.

As used herein, an “effective amount” or “therapeutically effective amount” refers to an amount of the compound of the present disclosure that is effective to achieve a desired therapeutic result such as, for example, improving the symptoms and/or decreasing the disease severity of an individual having cystic fibrosis, improving mucociliary clearance, increasing submucosal gland secretion, or suppressing cholinergic agonist induced muscle contractions. In the context of the present invention, a desired therapeutic result includes clearing mucus from the lungs in such patient or inhibiting mucus accumulation in such patient's lungs. While the doses mentioned in the present disclosure are guidelines, an attending physician may adjust the dose according to the specific needs of the patient, including for example, severity of the disease, size and physical condition.

Treat,” “treatment,” “prevent,” “prevention,” “inhibit” and corresponding terms include therapeutic treatments, prophylactic treatments, and ones that reduce the risk that a subject will develop a disorder or risk factor. Treatment does not require complete curing of disorder or condition, and includes the reduction in severity, reduction in symptoms, reduction of other risk factors associated with the condition and/or disease modifying effects such as slowing the progression of the disease.

Cystic fibrosis is an inherited disease that disrupts anion transport in exocrine glands and ‘wet’ epithelia and affects primarily the gastrointestinal and respiratory systems. It leads to chronic lung disease, exocrine pancreatic insufficiency, hepatobiliary disease, and abnormally high sweat electrolytes. Diagnosis is by sweat test or identification of 2 cystic fibrosis-causing gene variants in patients with a positive newborn screening test result or characteristic clinical features. Treatment is supportive through aggressive multidisciplinary care along with small-molecule correctors and potentiators targeting the cystic fibrosis transmembrane conductance regulator (CTFR) protein defect.

Cystic fibrosis is carried as an autosomal recessive trait by about 3% of the white population and variably lower carrier rates in non-white populations. The responsible gene has been localized on the long arm of chromosome 7. It encodes a membrane-associated protein called the cystic fibrosis transmembrane conductance regulator (CFTR). The most common gene variant, F508del, occurs in about 85% of CF alleles; >2000 less common CFTR variants have been identified.

CFTR is a cyclic adenosine monophosphate (cAMP)-regulated anion channel. It conducts chloride and bicarbonate, and influences transport of other ions, particularly sodium, across epithelial membranes. A number of additional functions are considered likely. Disease manifests only in homozygotes. Heterozygotes show subtle abnormalities of epithelial electrolyte transport and are largely clinically unaffected, but have small, increased risk for many CF-related conditions (Miller, Proc Natl Acad Sci U S A, 2020, 117, 1621).

CFTR variants have been divided into six classes based on how the variant affects the function or processing of the CFTR protein. Patients with class I, II, or III variants are considered to have a more severe genotype that results in little or no CFTR function, whereas patients with 1 or 2 class IV, V, or VI variants are considered to have a milder genotype that results in residual CFTR function. However, there is no strict relationship between specific variants and disease manifestation, so clinical testing (i.e., of organ function) rather than genotyping is a better guide to prognosis. CFTR variants can involve frameshift (a deletion or insertion in a DNA sequence that shifts the way a sequence is read) or nonsense (stop) mutations.

Fifty percent of patients not diagnosed through newborn screening present with pulmonary manifestations, often beginning in infancy. The pulmonary disease is a consequence of mucus obstructing the airways. This mucus obstruction promotes recurrent or chronic infections manifested commonly by cough, sputum production, and wheezing. Cough is the most troublesome complaint, often accompanied by sputum, gagging, vomiting, and disturbed sleep. Intercostal retractions, use of accessory muscles of respiration, a barrel-chest deformity, digital clubbing, cyanosis, and a declining tolerance for exercise occur with disease progression. Upper respiratory tract involvement includes nasal polyposis and chronic or recurrent rhinosinusitis.

β-adrenergic agonists are medications that relax muscles of the airways, causing widening of the airways and resulting in easier breathing. They are a class of sympathomimetic agents, each acting upon the beta adrenoceptors. In general, pure beta-adrenergic agonists have the opposite function of beta blockers: beta-adrenoreceptor agonist ligands mimic the actions of both epinephrine- and norepinephrine-signaling, in the heart and lungs, and in smooth muscle tissue; epinephrine expresses the higher affinity. The activation of β₁, β₂ and β₃ activates the enzyme, adenylate cyclase. This, in turn, leads to the activation of the secondary messenger cyclic adenosine monophosphate (cAMP); cAMP then activates protein kinase A (PKA) which phosphorylates target proteins, ultimately inducing smooth muscle relaxation and contraction of the cardiac tissue.

Cholinergic agonists are a category of pharmaceutical agents that act upon the neurotransmitter acetylcholine, the primary neurotransmitter within the parasympathetic nervous system (PNS). Cholinergic agonists stimulate cholinergic receptors which include nicotinic and muscarinic receptors. There are two broad categories of cholinergic drugs: direct-acting and indirect-acting. The direct-acting cholinergic agonists work by directly binding to and activating the muscarinic receptors. Examples of direct-acting cholinergic agents include choline esters (acetylcholine, methacholine, carbachol, bethanechol) and alkaloids (muscarine, pilocarpine, cevimeline). Indirect-acting cholinergic agents increase the availability of acetylcholine at the cholinergic receptors. These include reversible agents (physostigmine, neostigmine, pyridostigmine, edrophonium, rivastigmine, donepezil, galantamine) and irreversible agents (echothiophate, parathion, malathion, diazinon, sarin, soman).

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

In further describing the various aspects of the invention, methods for treating an individual for cystic fibrosis are described first in greater detail. Next, methods for increasing submucosal gland secretion are described. Next, methods for suppressing cholinergic agonist induced muscle contractions are described. Finally, compositions for practicing the methods disclosed herein are reviewed.

### Methods for Treating an Individual for a Muco-Obstructive Disorder

The present disclosure provides a method for treating an individual for a muco-obstructive disorder, the method including administering to the individual a β-adrenergic agonist or an adenylyl cyclase activator, in combination with a cholinergic agonist to treat the individual for the muco-obstructive disorder.

The methods disclosed herein may be used to treat a number of muco-obstructive disorders to either directly treat said disorders or to alleviate the symptoms associated with said disorders. Muco-obstructive disorders that may be treated with the methods include, without limitation, cystic fibrosis, primary ciliary dyskinesia, asthma, chronic obstructive pulmonary disease (COPD), idiopathic pulmonary fibrosis, chronic bronchitis, non-CF bronchiectasis, etc.

When the muco-obstructive disorder is cystic fibrosis the individual may be any individual who is predicted to have or has been diagnosed with cystic fibrosis. The diagnosis may be based on specific genetic variants in the CFTR gene resulting in a non-functional CTFR protein or a CFTR with reduced functionality. The diagnosis may also be based on a symptom or a collection of symptoms of clinical features. Genetic variants associated with cystic fibrosis include, without limitation, G85E, R117H, 621+1G→T, 711+1G→T, 1078delT, R334W, R347P, A455E, ΔI507,ΔF508, 1717-1G-A, G542X, S549N, G551D, R553X, R560T, 1898+1 G→A, 2184delA, 2789+5 G→A, R1162X, 3659delC, 3849+10kbC, W1282X, N1303K, etc. The individual may have genetic variants other than those listed above. The individuals that may be treated for cystic fibrosis or other muco-obstructive disorders using the methods are generally mammals. Non-limiting examples of mammals that may be treated using the methods include, without limitation, a pig, a ferret, a cow, a goat, a sheep, a rodent, a rat, a mouse, a non-human primate, a human, etc. In some embodiments, the individual is a human.

The β-adrenergic agonists of the present disclosure may be any β-adrenergic agonist that activates a β-adrenergic receptor. The-adrenergic agonist may target any β-adrenergic receptor deemed useful including a β₁-adrenergic receptor, a β₂-adrenergic receptor or a β₃-adrenergic receptor. In some embodiments, the β-adrenergic agonist is a β₂-adrenergic agonist that targets a β₂-adrenergic receptor. A number of different β₂-adrenergic agonist may be used to practice the methods disclosed herein including, without limitation, bitolterol, fenoterol, isoprenaline, isoproterenol, levosalbutamol, levalbuterol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, isoxsuprine, mabuterol, zilpaterol, etc. In some embodiments, the β₂-adrenergic agonist is formoterol. In some embodiments, an adenylate cyclase activator such as forskolin is used instead of a β₂-adrenergic. Adenylate cyclase activators include forskolin and colforsin.

Cholinergic agonists of the present disclosure are any molecule that mimics the activity of the neurotransmitter acetylcholine and act on muscarinic receptors. A range of different cholinergic agonists may be utilized in the methods practiced herein. The cholinergic agonist may be direct acting or indirect acting. Non-limiting examples of direct acting cholinergic agonists are acetylcholine, methacholine, carbachol, bethanechol, muscarine, pilocarpine, and cevimeline. Non-limiting examples of indirect acting cholinergic agonists are physostigmine, neostigmine, pyridostigmine, edrophonium, rivastigmine, donepezil, galantamine, echothiophate, parathion, malathion, diazinon, sarin, and soman. In some embodiments, the direct acting cholinergic agonist is methacholine. In some embodiments, the direct acting cholinergic agonist is carbachol.

The combination of the β-adrenergic agonist or the adenylyl cyclase activator, and the cholinergic agonist may be administered in a specific timing and/or a specific order in order to treat the individual for cystic fibrosis or other muco-obstructive disorders. As such, the β-adrenergic agonist or the adenylyl cyclase activator, and the cholinergic agonist may be administered sequentially or simultaneously. In some embodiments, the β-adrenergic agonist or the adenylyl cyclase activator is administered and then the cholinergic agonist is administered after a delay. In some embodiments, the cholinergic agonist is administered and then the β-adrenergic agonist, or the adenylyl cyclase activator is administered after a delay. The delay may be a range of different periods. For instance, the delay may be at 5 minutes or longer, 10 minutes or longer, 15 minutes or longer, 20 minutes or longer, 25 minutes or longer, 30 minutes or longer, 35 minutes or longer, 40 minutes or longer, 45 minutes or longer, 50 minutes or longer, 55 minutes or longer, 60 minutes or longer, or greater than about 60 minutes.

In some embodiments, the combination of the β-adrenergic agonist or the adenylyl cyclase activator, and the cholinergic agonist are administered simultaneously. When the combination of the β-adrenergic agonist of the adenylyl cyclase activator, and the cholinergic agonist are administered simultaneously or when the β-adrenergic agonist or the adenylyl cyclase activator is administered before the cholinergic agonist a particular benefit may be conferred. For instance, simultaneous administration or administration of the β-adrenergic agonist or the adenylyl cyclase activator prior to the cholinergic administration can suppress cholinergic agonist induced airway smooth muscle contractions. Prior to the present disclosure it was well established that the β-adrenergic agonist is not to be administered prior to the cholinergic agonist. It was unexpectedly found that administering the β-adrenergic agonist prior to the cholinergic agonist can suppress cholinergic agonist-induced airway smooth muscle contractions and airway narrowing. In some embodiments, the administration of the combination of the β-adrenergic agonist or the adenylyl cyclase activator, and the cholinergic agonist results in a synergistic increase in mucus transport relative to the mucus transport of either agonist alone.

The methods involve administering the β-adrenergic agonist or the adenylyl cyclase activator, in combination with the cholinergic agonist locally or systemically. When the combination is administered locally, the combination may be administered directly to the trachea or the lungs or at a site near the trachea or lungs. When the combination is administered locally, the combination may be administered using an oral or nasal inhaler. When the combination is administered systemically, the combination may be administered in a convenient systemic form, such as in the form of a pill or oral tablet.

The combination of the β-adrenergic agonist or the adenylyl cyclase activator, and the cholinergic agonist may be administered as a single daily dose, multiple doses per day (e.g., 2 or 3 doses per day), intermittent, or weekly, with the dosing regimen dependent on the dosage form (e.g., immediate release or controlled release), and the needs of the individual. Administration may be for an extended period of time, intermittent, or may be for a limited amount of time, with administration repeated if and to the extent determined by a qualified professional. For example, the combination may be provided daily for 1 day, 2 days, 3 days, 4 days, 5 days, 6 days, 7 days, 8 days, 9 days, 10 days, 12 days, 14 days, 16 days, 18 days, 20 days, 22 days, 24 days, 26 days, 28 days, 30 days, or greater than 30 days and then stopped. In some embodiments, the combination is administered intermittently, such as every 2 or 3 days, or weekly.

In some embodiments, the methods further include administering one or more cystic fibrosis transmembrane conductance regulator (CFTR) modulators. It has been found that combination of the β-adrenergic agonist or the adenylyl cyclase activator, and the cholinergic agonist administered in combination with a CFTR modulator is effective in increasing mucus clearance. When a CFTR modulator is administered in conjunction with the combination of the β-adrenergic agonist or the adenylyl cyclase activator, and the cholinergic agonist, any CFTR modulator may be used that is effective in treating cystic fibrosis. CFTR modulators that find use in the present disclosure include, without limitation, Elexacaftor, Tezacaftor, Ivacaftor, Lumacaftor, Vanzacaftor, Deutivacaftor, etc. In an embodiment, the one or more CFTR modulators are Elexacaftor, Tezacaftor, and Ivacaftor. In some embodiments, the administration of the combination of the β-adrenergic agonist or the adenylyl cyclase activator, and the cholinergic agonist in conjunction with the one or more CFTR modulations results in a synergistic increase in mucus transport relative to the mucus transport of either the combination or the one or more CFTR modulators alone.

In some embodiments, the combination of the β-adrenergic agonist or the adenylyl cyclase activator, and the cholinergic agonist may be administered with other medicants other than or in addition to the one or more CFTR modulators. Other medicants include any medicant that directly treats cystic fibrosis or other muco-obstructive disorders, or the symptoms thereof such as substances that improve mucus clearance. For instance, other medicants include, without limitation, recombinant human DNases such as Pulmozyme, hypertonic saline, powered mannitol, etc., as well as vectors designed to transfect airway cells with agents designed to augment the defective CFTR protein by supplying reagents such as cDNA or mRNA.

The treatments disclosed herein may have a number of different effects on individuals having cystic fibrosis or other muco-obstructive disorders. For instance, the administration may increase mucociliary clearance velocity, increase the rate of airway submucosal gland secretion, suppress cholinergic agonist induced airway smooth muscle contractions. The administration may also reduce symptoms associated with cystic fibrosis or other muco-obstructive disorders including, without limitation, recurrent or chronic infection, coughing, sputum production, wheezing, abdominal distension, constipation, etc.

### Methods for Increasing the Rate of Airway Submucosal Gland Secretion

The present disclosure provides a method for increasing airway submucosal gland secretion in an individual, the method including administering to the individual a β-adrenergic agonist or the adenylyl cyclase activator, in combination with a cholinergic agonist to increase the rate of airway submucosal glad secretion in the individual.

The β-adrenergic agonists of the present disclosure may be any β-adrenergic agonist that activates a β-adrenergic receptor. In an embodiment, the β-adrenergic agonist is a β₂-adrenergic agonist that targets a β₂-adrenergic receptor. A number of different β₂-adrenergic agonist may be used to practice the methods disclosed herein including, without limitation, bitolterol, fenoterol, isoprenaline, isoproterenol, levosalbutamol, levalbuterol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, isoxsuprine, mabuterol, zilpaterol, etc. In some embodiments, the β₂-adrenergic agonist is formoterol. In some embodiments, an adenylate cyclase activator such as forskolin is used instead of a β₂-adrenergic agonist.

Cholinergic agonists of the present disclosure are any molecule that mimics the activity of the neurotransmitter acetylcholine and act on muscarinic receptors. The cholinergic agonist may be direct acting or indirect acting Non-limiting examples of direct acting cholinergic agonists are acetylcholine, methacholine, carbachol, bethanechol, muscarine, pilocarpine, and cevimeline. Non-limiting examples of indirect acting cholinergic agonists are physostigmine, neostigmine, pyridostigmine, edrophonium, rivastigmine, donepezil, galantamine, echothiophate, parathion, malathion, diazinon, sarin, and soman. In some embodiments, the cholinergic agonist is methacholine. In some embodiments, the cholinergic agonist is carbachol.

The combination of the β-adrenergic agonist or the adenylyl cyclase activator, and the cholinergic agonist may be administered in a specific timing and/or a specific order in order to treat the individual for cystic fibrosis. In some embodiments, the β-adrenergic agonist or the adenylyl cyclase activator is administered and then the cholinergic agonist is administered after a delay. In some embodiment, the combination of the β-adrenergic agonist or the adenylyl cyclase activator, and the cholinergic agonist are administered simultaneously.

The methods disclosed herein increase the rate of airway submucosal gland secretion. The rate of submucosal gland secretion can be increased by a range of different values. For instance, the administration of the combination of a β-adrenergic agonist and cholinergic agonist may increase the rate of submucosal gland secretion by at least about 0.5 nL per minute, at least about 1.0 nL per minute, at least about 1.5 nL per minute, at least about 2 nL per minute, at least about 3 nL per minute, at least about 4 nL per minute, at least about 5 nL per minute, at least about 6 nL per minute, at least about 7 nL per minute, at least about 8 nL per minute, at least about 9 nL per minute, at least about 10 nL per minute, or greater than about 10 nL per minute.

The rate of submucosal gland secretion may be increased in a variety of different individuals. Individuals that may receive particular benefit from such methods include individuals who have reduced production of mucus, individuals who produce overly viscous mucus or individuals having chronic airway inflammation. The individuals that may respond to the methods are generally mammals. Non-limiting examples of mammals that may be treated using the methods include, without limitation, a pig, a ferret, a cow, a goat, a sheep, a rodent, a rat, a mouse, a non-human primate, a human, etc. In some embodiments, the individual is a human. In some embodiments, the individual is a pig. In some embodiments, the individual is a ferret.

In some embodiments, the individual has a genetic variant of CFTR. Genetic variants of CFTR include, without limitation, G85E, R117H, 621+1G→T, 711+1G→T, 1078delT, R334W, R347P, A455E, ΔI507, ΔF508, 1717-1G-A, G542X, S549N, G551D, R553X, R560T, 1898+1 G→A, 2184delA, 2789+5 G→A, R1162X, 3659delC, 3849+10kbC, W1282X, N1303K, etc. The individual may have genetic variants other than those listed above.

### Methods for Suppressing Cholinergic Agonist Induced Airway Smooth Muscle Contraction

The present disclosure provides a method for suppressing cholinergic agonist induced airway smooth muscle contraction in an individual, the method including administering to the individual a β-adrenergic agonist or an adenylyl cyclase activator, wherein the administration of the β-adrenergic agonist or the adenylyl cyclase activator occurs before or at the same time that the individual has been administered a cholinergic agonist to suppress the airway smooth muscle contraction.

The β-adrenergic agonists of the present disclosure may be any β-adrenergic agonist that activates a β-adrenergic receptor. In an embodiment, the β-adrenergic agonist is a β₂-adrenergic agonist that targets a β₂-adrenergic receptor. A number of different β₂-adrenergic agonist may be used to practice the methods disclosed herein including, without limitation, bitolterol, fenoterol, isoprenaline, isoproterenol, levosalbutamol, levalbuterol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, isoxsuprine, mabuterol, zilpaterol, etc. In some embodiments, the β₂-adrenergic agonist is formoterol. In some embodiments, an adenylate cyclase activator such as forskolin is used instead of a β₂-adrenergic agonist

Cholinergic agonists of the present disclosure are any molecule that mimics the activity of the neurotransmitter acetylcholine and act on muscarinic receptors. The cholinergic agonist may be direct acting or indirect acting. Non-limiting examples of direct acting cholinergic agonists are acetylcholine, methacholine, carbachol, bethanechol, muscarine, pilocarpine, and cevimeline. Non-limiting examples of indirect acting cholinergic agonists are physostigmine, neostigmine, pyridostigmine, edrophonium, rivastigmine, donepezil, galantamine, echothiophate, parathion, malathion, diazinon, sarin, and soman. In some embodiments, the cholinergic agonist is methacholine. In some embodiments, the cholinergic agonist is carbachol.

The methods disclosed herein suppress cholinergic agonist induced airway smooth muscle contractions. The cholinergic agonist induced airway smooth muscle contractions can be suppressed by a range of different amounts. For instance, the administration of the β-adrenergic agonist or the adenylyl cyclase activator may suppress cholinergic agonist induced muscle contractions by at least about 50%, at least about 55%, at least about 60%, at least about 65%, at least about 70%, at least about 75%, at least about 80%, at least about 85%, at least about 90%, at least about 95%, or by about 100%.

Cholinergic agonist induced airway smooth muscle contraction may be suppressed in a range of different individuals. Individuals that may receive particular benefit from such methods are individuals who have taken or regularly take cholinergic agonist based medications. Cholinergic agonist based medications are used to treat a number of different disorders including, without limitation, Myasthenia Gravis, Xerostomia, Urinary retention, Neurogenic bladder, Eye surgery adjunct, Glaucoma, Dementia, acute colonic pseudo-obstruction, Anticholinergic overdose, Sjogren's Syndrome, etc. The individuals that may respond to the methods are generally mammals. Non-limiting examples of mammals that may be treated using the methods include, without limitation, a pig, a ferret, a cow, a goat, a sheep, a rodent, a rat, a mouse, a non-human primate, a human, etc. In some embodiments, the individual is a human. In some embodiments, the individual is a pig. In some embodiments, the individual is a ferret.

In some embodiments, the individual has a genetic variant of CFTR. Genetic variants of CFTR include, without limitation, G85E, R117H, 621+1G→T, 711+1G→T, 1078delT, R334W, R347P, A455E, ΔI507, ΔF508, 1717-1G-A, G542X, S549N, G551D, R553X, R560T, 1898+1 G→A, 2184delA, 2789+5 G→A, R1162X, 3659delC, 3849+10kbC, W1282X, N1303K, etc. The individual may have genetic variants other than those listed above.

### Compositions

Also compositions for practicing the methods are described in the present disclosure. In general, subject compositions may have the β-adrenergic agonist or the adenylyl cyclase activator, and the cholinergic agonist as described above in addition to a pharmaceutical excipient as described above. The composition may be contained in a device. In some embodiments the device is an inhaler.

In some embodiments, the composition may further include one or more CFTR modulators. CFTR modulators of the present disclosure have been described above in greater detail. In some embodiments, the composition may further comprise other medicants other than or in addition to the one or more CFTR modulators. Other medicants include any medicant that directly treats cystic fibrosis or other muco-obstructive disorders, or the symptoms thereof such as substances that improve mucus clearance. For instance, other medicants include, without limitation, recombinant human DNases such as Pulmozyme, hypertonic saline, powered mannitol, etc., as well as vectors designed to transfect airway cells with agents designed to augment the defective CFTR protein by supplying reagents such as cDNA or mRNA.

In some embodiments, the composition is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the composition may further include a preservative. Suitable preservatives include, but are not limited to, a benzyl alcohol, phenol, chlorobutanol, benzalkonium chloride, and the like. In many cases, the formulation is stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The composition may be administered in a unit dosage form and may be prepared by any methods well known in the art. Such methods include combining the β-adrenergic agonist or the adenylyl cyclase activator, and the cholinergic agonist with a pharmaceutically acceptable excipient or diluent which constitutes one or more accessory ingredients. A pharmaceutically acceptable excipient is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

The combination of the β-adrenergic agonist or the adenylyl cyclase activator, and the cholinergic agonist may be administered by pharmaceutical dosage forms known in the art, including but not limited to oral solid dosages, oral liquids, injection, transdermal patch, and inhalation. Oral dosages are formulated for systemic delivery while inhalation dosages are formulated for local delivery. Systemic formulations are meant to be digested in the stomach and intestines while inhalation formulations are meant for local direct delivery to the affected tissues including the trachea and the lungs. Dosage forms may be formulated with excipients and other compounds to facilitate administration to a subject and to maintain shelf stability. See “Remington's Pharmaceutical Sciences” (Mack Publishing Co., Easton, PA). Oral pharmaceutical formulations include tablets, minitablets, pellets, granules, capsules, gels, liquids, syrups and suspensions. The combination may be administered orally, typically via oral solid dosage, although oral liquids may be desirable for certain populations that have difficulty with tablets and capsules, such as pediatric and elderly patients. Oral dosage forms may be immediate release or controlled release.

In one embodiment of the invention, the combination of the β-adrenergic agonist or the adenylyl cyclase activator, and the cholinergic agonist may be provided as an immediate release formulation. Immediate release of the combination may be provided as a single daily dose, or divided into multiple daily dosages which may be administered 2, 3, 4 or more times per day. In another embodiment of the invention, the combination is provided as an extended release formulation. An extended release formulation may provide patient convenience by reducing daily administrations, and may improve patient compliance. Further, controlled release formulations of the present invention may be useful in reducing serum peaks and troughs, thereby potentially reducing adverse events.

Oral controlled release formulations are known in the art and include sustained release, extended release, delayed release and pulsatile release formulations. See “Remington's Pharmaceutical Sciences” (Mack Publishing Co., Easton, PA). The active agent may be formulated in a matrix formulation with one or more polymers that slow release of the drug from the dosage form, including hydrophilic or gelling agents, hydrophobic matrices, lipid or wax matrices and biodegradable matrices. The active agent may be formulated in the form of a bead, for example with an inert sugar core, and coated with known excipients to delay or slow release of the active agent by diffusion. Enteric coatings are known in the art for use in delaying release of an active agent until the dosage form passes from the low pH environment of the stomach to the higher pH environment of the small intestine, and my include methyl acrylate-methacrylic acid copolymers, cellulose acetate phthalate (CAP), cellulose acetate succinate, hydroxypropyl methylcellulose phthalate, hydroxypropyl methylcellulose acetate succinate, polyvinyl acetate phthalate (PVAP), shellac, sodium alginate, and cellulose acetate trimellitate.

The compositions of the present disclosure may be contained within a device. The device may be any device that allows for the composition to be delivery to the affected tissues such as the trachea or the lungs. The device includes, without limitation, a nebulizer, an oral inhaler, a nasal inhaler, etc. When the device is an inhaler, the inhaler may disperse a dry powder or a liquid in the form of an aerosolized spray. Aerosolized sprays are generally presented from pressurized packs with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler may be formulated containing a powder mix of the compound and a suitable powder base such as lactose or starch. As an example, preparations for administration by inhalation may be prepared according to the teaching of Quay, et al., U.S. Pat. No. 7,812,120 B2.

### Kits

Kits for use in practicing certain methods described herein are also provided. In certain embodiments, the kits include a β-adrenergic agonist and a cholinergic agonist, e.g., as described above. In a given kit, the active agents may be individually packaged or present in a formulation, e.g., where the active agents are delivered simultaneously to a subject. Active agents may be present in the same, or separate, containers, as desired.

In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. Yet another form of these instructions is a computer readable medium, e.g., diskette, compact disk (CD), portable flash drive, USB storage, DVD, Blu-ray disk, etc.), and the like, on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Synergistic increases of MCCV in CF ferrets and WT pigs. The ‘synergy paradigm’ is defined as the sequential exposure to 30 min of either a cAMP or Ca²⁺ elevating agonist, followed by at least a 30 min exposure to the combined agonists. 10 μM forskolin, isoproterenol or formoterol was used as cAMP agonists, and 0.3 μM carbachol as the Ca²⁺ elevating agonist (all basolateral). In prior work these combinations caused increases in MCCV of ferret tracheas that were much greater than the predicted additive effects of the two agonists and approached maximal values (16).

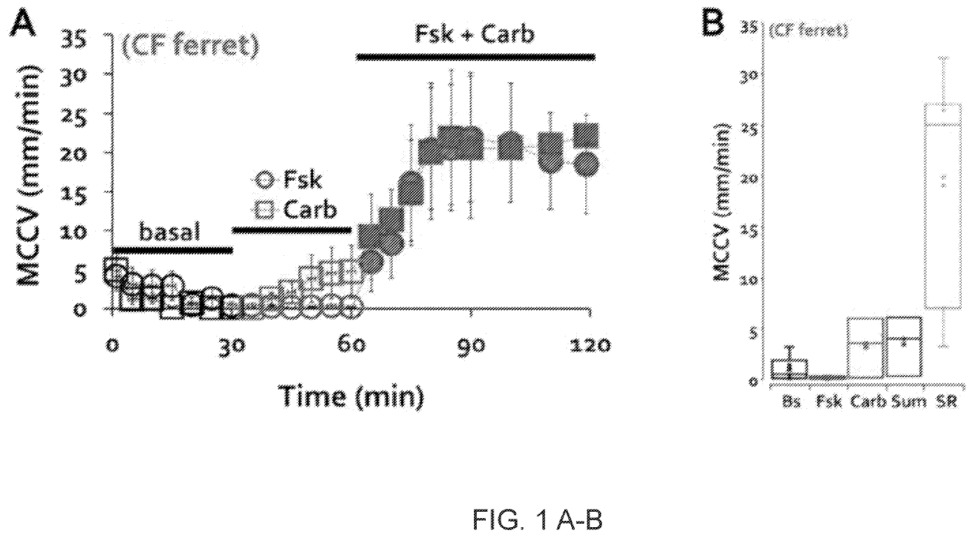

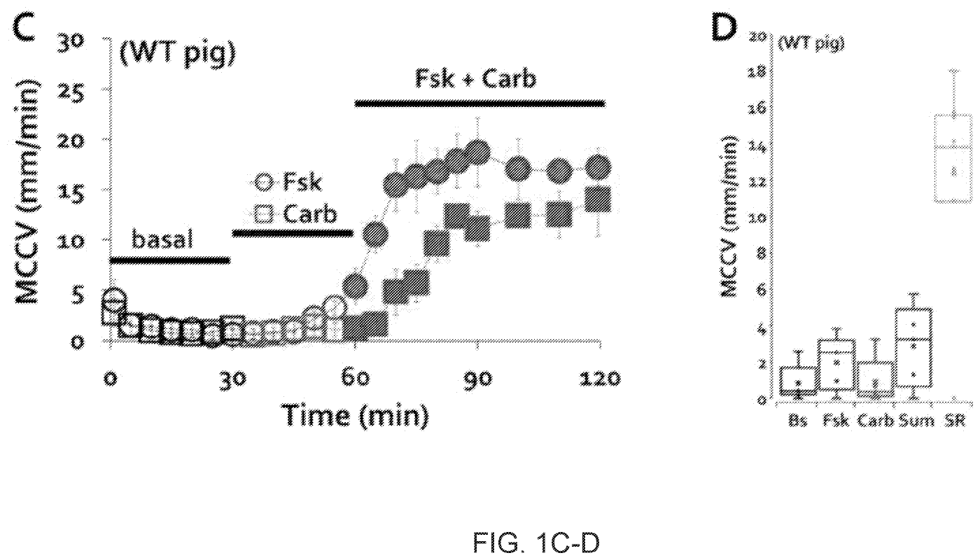

To determine if synergistic increases of MCCV could be produced in CF ferret tracheas, tracheas from 7 transgenic adult CF ferrets of mixed genotype were tested (see “Methods” section). The 7 CF ferret tracheas were divided into two groups and tested, 4 by applying forskolin first and 3 by applying carbachol first, followed by the combined agonists. MCCV (all values are in mm/min) was then measured for 90-150 min and plotted as MCCV vs time and agonist(s) in FIG. 1A. MCCV declined to near zero in the first 30 min without stimulation (FIG. 1A, see figure legend for details). Forskolin produced no increase in MCCV and carbachol produced only a small increase. However, when the agonists were then combined in either order, they produced large, sustained increases in MCCV to ˜20 mm/min. Averaged data for the last 20 min of each period of basal and single drug treatment and the period from 10 to 80 min after adding the combined agonists are shown as a box and whisker plot in FIG. 1B. MCCV values were: unstimulated: 1.6±1.09 (n=3); by 10 μM forskolin: 0.18±0.09 (n=4); by 0.3 μM carbachol: 3.29±2.08 (n=3), ‘Sum’ is the arithmetic sum of MCCV induced by the two agonists used separately: 3.5±2.07; SR, synergy response by the combined agonists: 19.95±4.12, (P=0.006 SR vs. Sum).

Encouraged by these results, it was then asked if synergy could be observed in different species. MCCV in tracheas was measured from 2 to 5 days old WT piglets (see “Methods” section for details). Unstimulated MCC velocity was less than 1 mm/min (averaged T10-30 MCCV, 0.93±0.39, n=8 piglet tracheas) like that of WT ferrets (16). The 8 piglet tracheas were divided into two groups and tested 4 by applying forskolin first and 4 by applying carbachol first, followed by the combined agonists. Carbachol or forskolin alone produced only small increases in MCCV, but the combined agonists produced large, sustained increases in MCCV to 12-17 mm/min regardless of the order of addition (FIG. 1C). Averaged data for the last 20 min of each period of basal and single agonist treatment and the last 50 min of synergy paradigm period are shown as a box and whisker plot in FIG. 1D. Averaged MCCV values were: by 0.3 μM carbachol: 0.91±0.63 to 1.12±0.82 (P=0.32, Carb vs. Bs/basal, 4 piglets); by 10 μM forskolin: 0.95±0.65 to 2.46±0.68 (P=0.13, n=4); sum of individual response (Sum): 3.58±1.06, and by the synergy paradigm (SR): 13.92±0.94.

The box and whisker plots make it clear that the increases in MCCV to the combined agonists were significantly larger for the combined agonists than for the arithmetic sum of the responses for both CF ferrets and WT pigs. Thus, MCCV synergy exists in at least two species and persists, at least in part, after loss of CFTR function. The MCCV of CF ferrets in the synergy condition was 5.7-fold faster than the arithmetically summed responses of agonists used separately, but the value of practical importance is how this compares to WT ferrets. The CF ferret values were compared with those obtained previously from WT ferrets (16). For forskolin alone, WT versus CF ferret MCCV values were 6.75±0.84 (n=28) versus 0.18±0.09 (n=4). For carbachol alone WT versus CF ferret MCCV values were 8.24±0.82 (n=12) versus 3.29±2.08 (n=3). For synergistic responses to the combined agonists, WT vs CF MCCV values were 36.24±0.9, (n=40) versus 19.95±4.12 (n=7). Thus, compared to WT ferrets, CF ferret responses were ˜0% for forskolin, ˜40% for carbachol, and ˜55% for synergy.

These experiments used forskolin to elevate cAMP. To evaluate a clinically readily available β-adrenergic drug, MCCV was measured in response to 10 μM of the β2-adrenergic receptor agonist formoterol in place of forskolin. Comparable synergistic increases in MCCV were seen in pig tracheas (in mm/min): baseline, 0.3±0.1 (n=12); 10 μM formoterol, 2.4±0.9 (n=5), 0.3 M carbachol, 1.3±0.8 (n=3); and 10.9±0.8 by the combined agonists (n=7 piglet tracheas) (FIG. 1E, F).

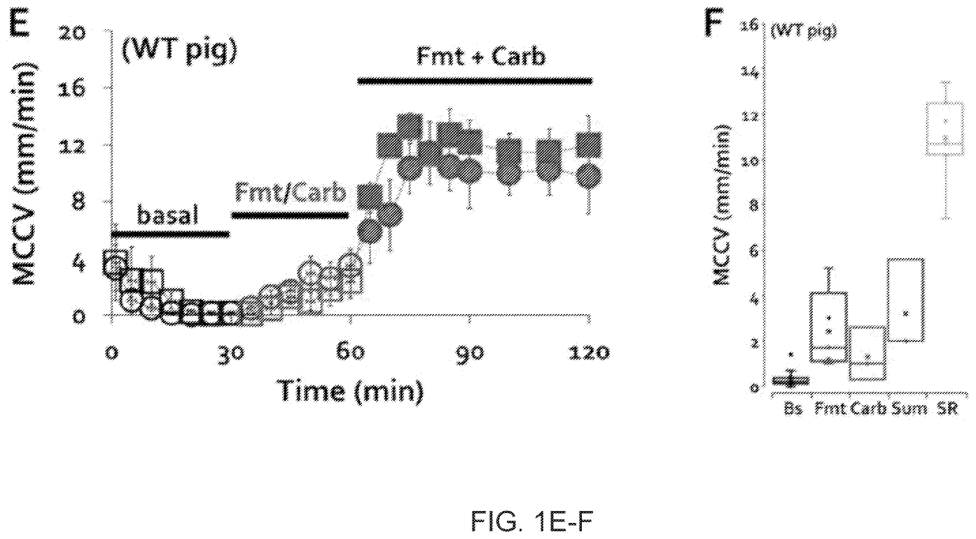

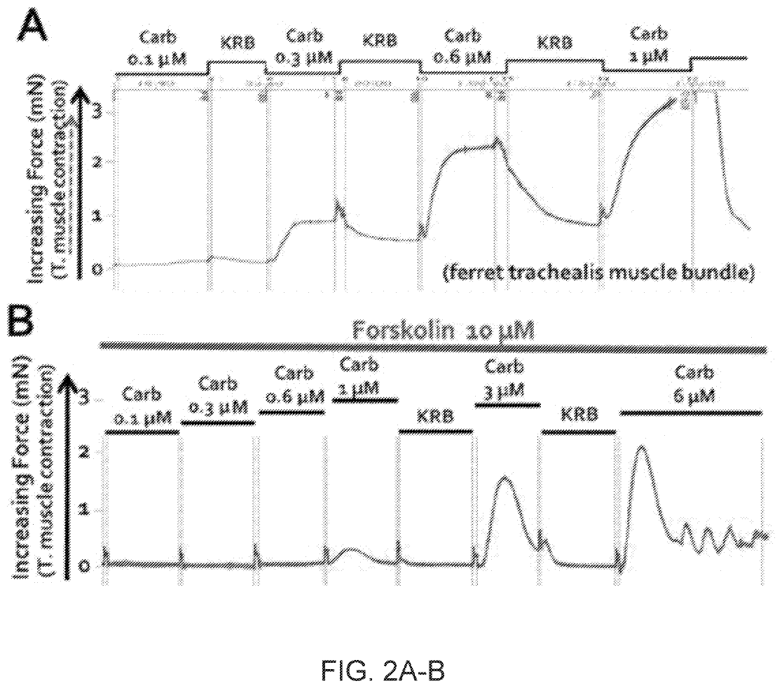

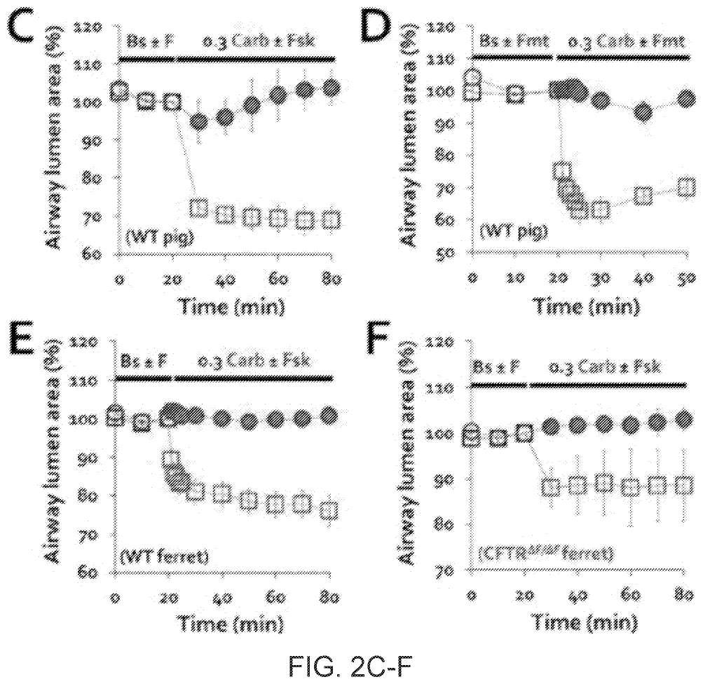

Combined agonists did not induce airway smooth muscle contraction or airway narrowing. The cAMP and Ca²⁺-elevating agonists that increase MCCV also affect airway smooth muscle. Used alone they have opposite effects: Ca²⁺-elevating agonists contract muscles whereas cAMP-elevating agonists relax them. For therapeutic use, the potential for producing unwanted bronchoconstriction with the combined agonists is a safety concern. To determine which effect predominates, airway smooth muscle responses to carbachol±10 mM forskolin or formoterol was measured using two different methods: measuring muscle tension and lumen area. Tension of ferret trachealis muscle bundles was measured to increasing carbachol concentrations±10 mM forskolin. Forskolin abolished tension increases to 0.3 and 0.6 mM carbachol and greatly reduced responses to higher doses of carbachol (FIG. 2A, B). Lumen area, imaged in thin sliced piglet or ferret tracheal rings, displayed a sustained 20-40% reduction with exposure to 0.3 mM carbachol, but when carbachol was preceded by either forskolin or formoterol, it induced only a transient decrease of 5% or less (FIG. 2C-F). Importantly, the protective effect was also observed in CF ferrets (FIG. 2F).

The velocity of mucus clearance reflects the transportability of mucus and ciliary beat frequency. Transportability is in turn largely determined by hydration/concentration (17) and pH (or bicarbonate content) of the mucus (18,19). A major source of upper airway fluid is submucosal glands, and the agonists used to stimulate MCCV also stimulate submucosal gland secretion (20-29). ASL depth and composition are also modified by surface epithelia that secrete and absorb electrolytes/fluid. Indeed, this is the principle means of controlling ASL in airways that lack submucosal glands (30). In prior work by us and others (26, 31-33)) evidence was found for cholinergic inhibition of Na⁺ absorption, which would tend to increase the fluidity and transportability of mucus. Finally, CBF is increased by elevating either Ca²⁺ (34) or cAMP (35). The following experiments sought evidence to support or challenge a possible contribution of each of these mechanisms to synergistic increases in MCCV.

Synergistic glandular mucus secretion in WT pigs, WT ferrets, and CF ferrets. Synergistic increases in MCCV rely partly on increased mucus secretion from submucosal glands. This hypothesis arises from evidence that combinations of [Ca²⁺]ᵢ-elevating and [cAMP]ᵢ-elevating agonists produce synergistically elevated rates of mucus from submucosal glands of humans (22), pigs (24), and ferrets (21). However, in those experiments different specific concentrations of agonists were used. To determine if the same protocols used here produced synergistic increases in secretion from submucosal glands, mucus secretion rates of individual tracheal glands in WT pigs and ferrets and in CF ferrets were measured via time-lapse optical imaging (28) while stimulating them with the same concentrations of agonists and durations of exposure used for MCCV studies. All secretion rates are reported as nanoliters/min/gland.

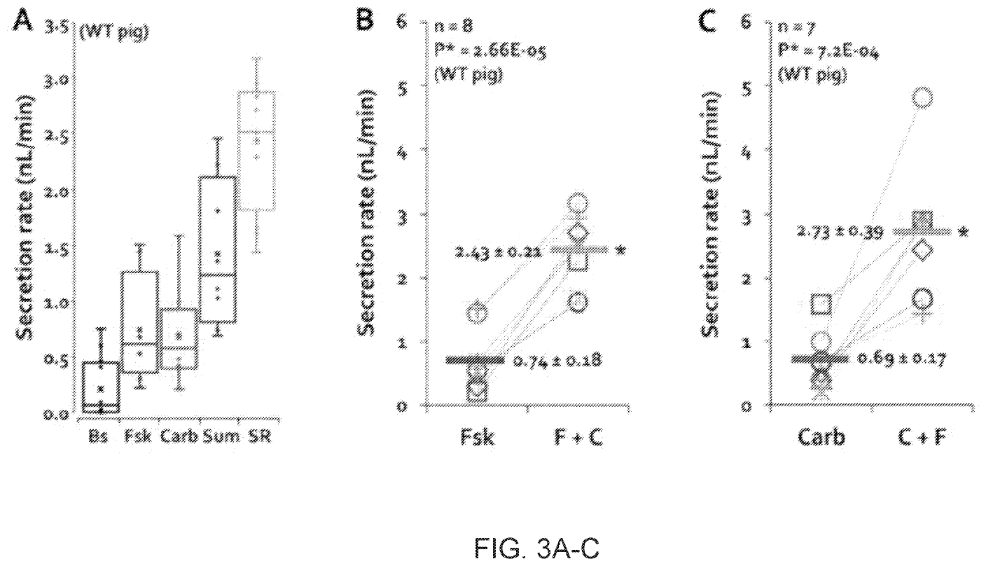

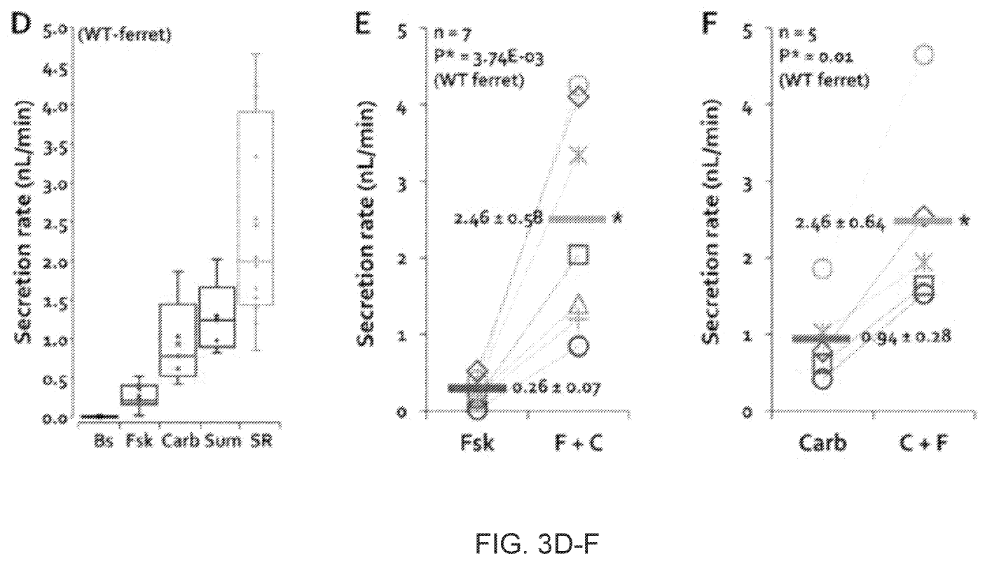

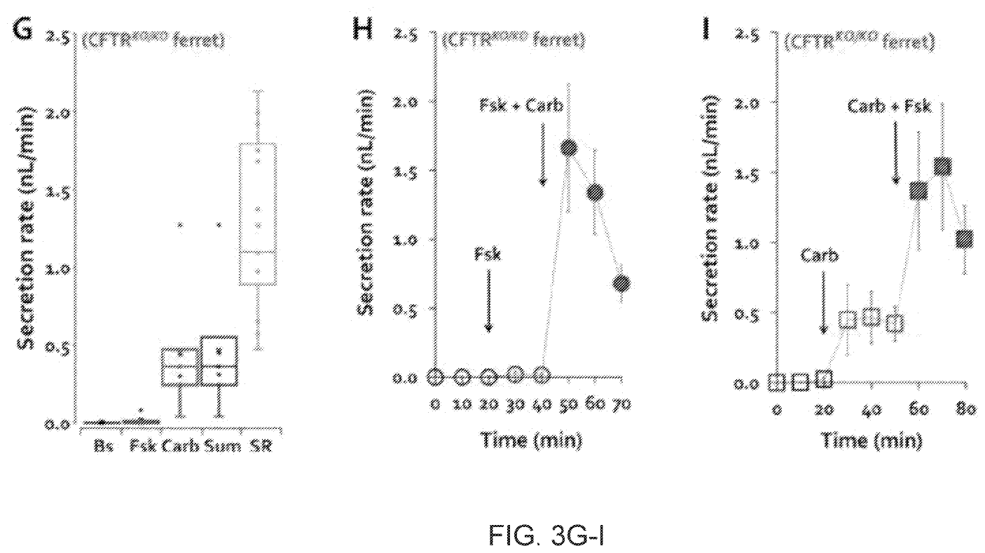

In WT pig tracheal glands, the average unstimulated secretion rate was in nl/min/gland, (0.21±0.06, 121 glands, 8 pigs, FIG. 3A). The basal rate was significantly increased by each agonist alone and was further increased by their combination in either order. Rates to the combined agonists were significantly larger than the arithmetic sum of their individual responses: additive sum=1.26±0.19, 7 pigs versus combined agonists=2.86±0.25 (2.3-fold larger, P<0.01, 8 pigs). Data for individual pigs is shown in FIG. 3B for forskolin first and in FIG. 3C for carbachol first.

WT Ferrets gave similar results (FIG. 3D-F). In ferret tracheal glands, the average unstimulated secretion rate was ˜zero (0.003±0.001, 67 glands, 7 ferrets, FIG. 3D). It was significantly increased by forskolin (0.26±0.07, 37 glands, 7 ferrets P<0.05) and by carbachol (0.94±0.28, 30 glands, 7 ferrets P<0.05). The secretion rates to the combined agonists were significantly larger than the arithmetical sum of their individual responses (FIG. 3D): overall arithmetic sum=1.27±0.23 versus 2.46±0.39 for the combined agonists (1.9-fold larger, P<0.05, 55-67 glands, 5-7 ferrets. Data for individual WT control ferrets is shown in FIG. 3E for forskolin first and in FIG. 3F for carbachol first.

Importantly, CF ferrets (CFTRKO/KO) also showed synergistic gland secretion in spite of having no response to forskolin alone. Only two CF ferrets were able to be tested (FIG. 3G-I). One CF ferret was stimulated first with 10 mM forskolin, the other with 0.3 mM carbachol, and both with the synergy paradigm. Unstimulated secretion rates were ˜zero, as in WT ferrets. Forskolin alone failed to stimulate secretion as expected (0.01, 7 glands), carbachol alone increased the average secretion rate to 0.45±0.16, and the combined agonists increased average rates to 1.23±0.35, 7 glands, and 1.31±0.19, 7 glands. When agonists were combined synergy was seen in both orders of addition. The averaged secretion rate across both ferrets to the combined agonists was 1.27±0.15,which is 2.8 times the arithmetic sum of the two agonists used alone, and about half of the response of WT ferrets of 2.46±0.39 (16).

To summarize this section, the rates of mucus secretion across both species and including CF ferrets is increased to values beyond the additive sum of the agents used alone, providing circumstantial evidence that gland secretion rates contribute to MCCV in our system.

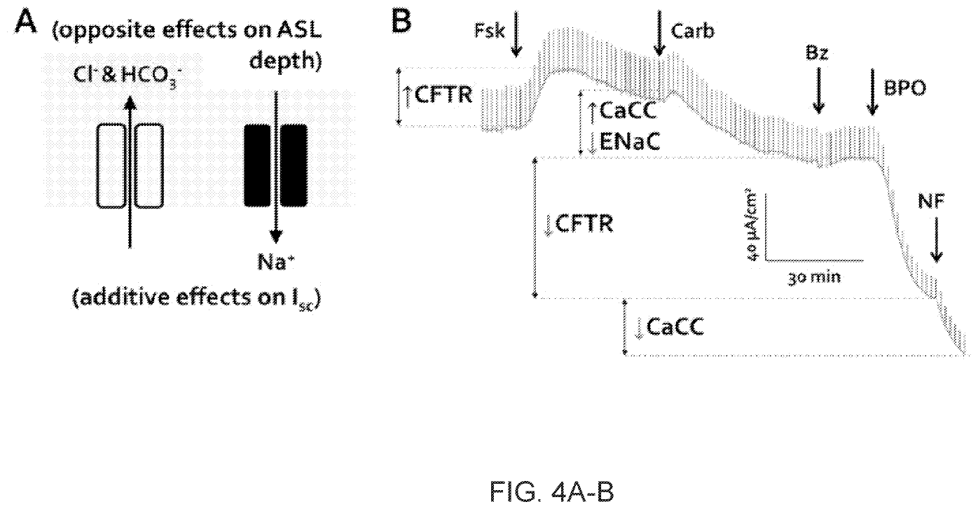

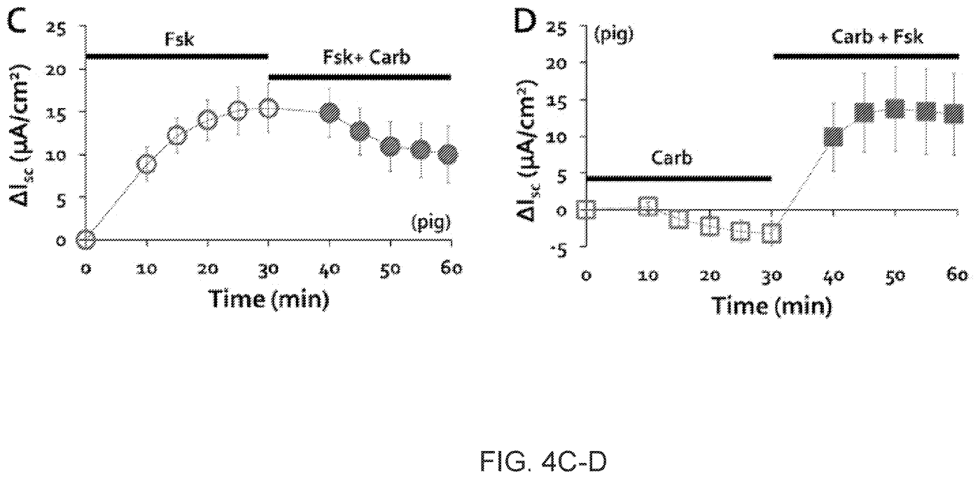

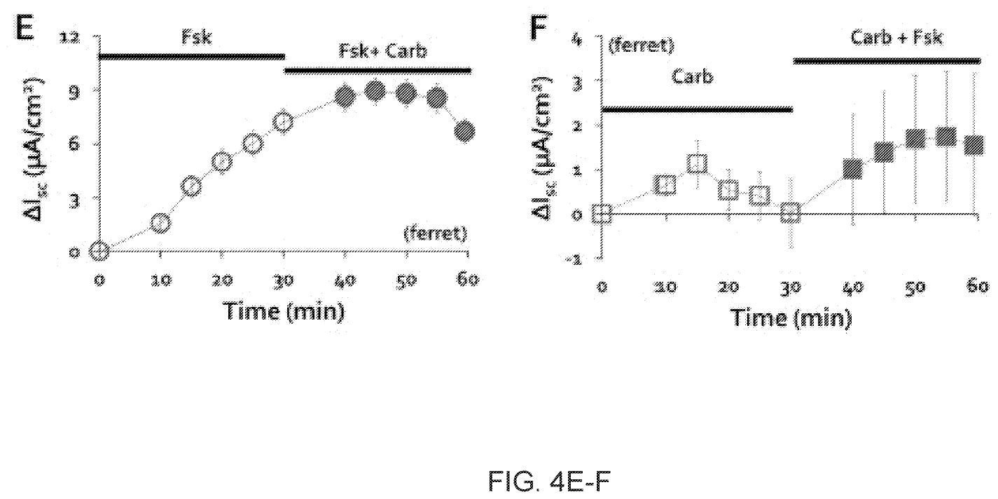

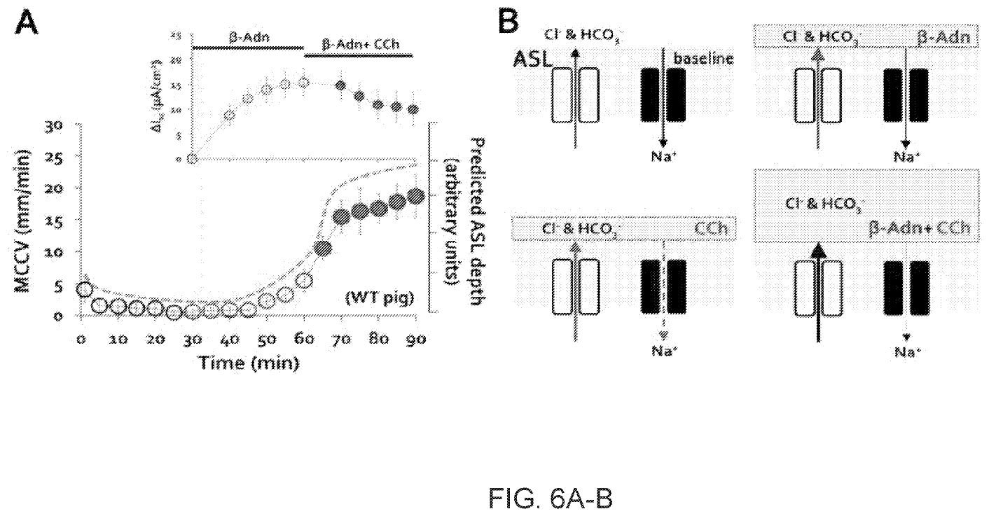

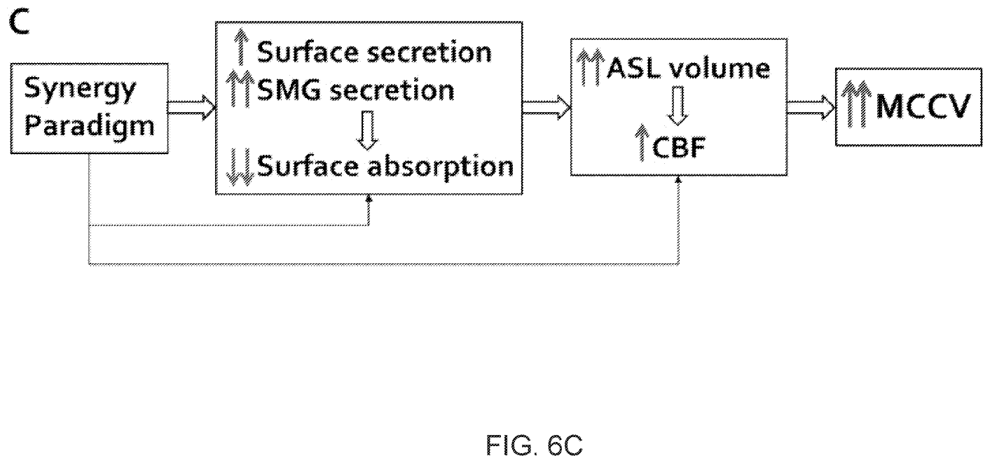

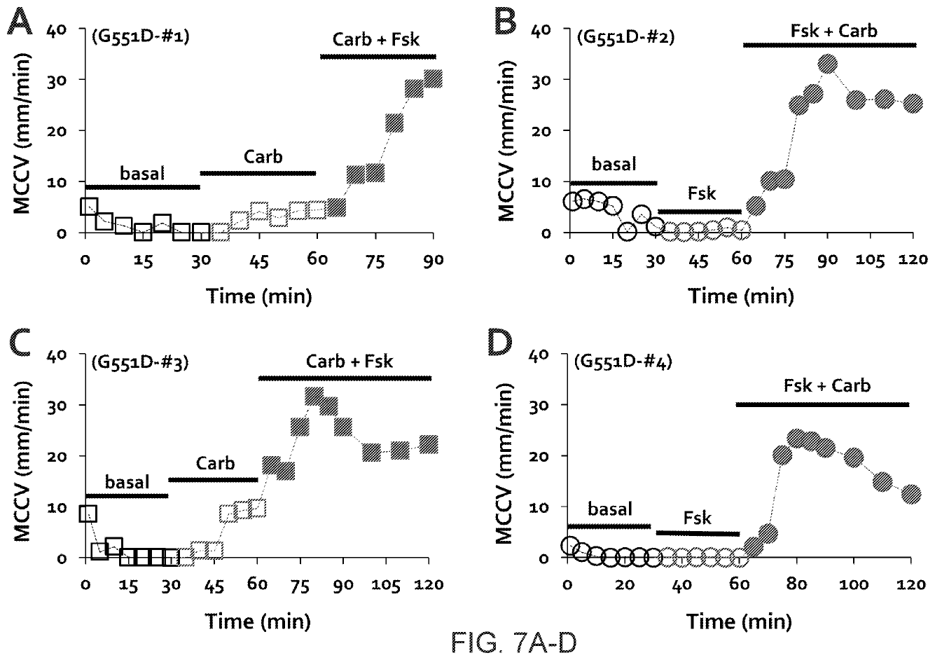

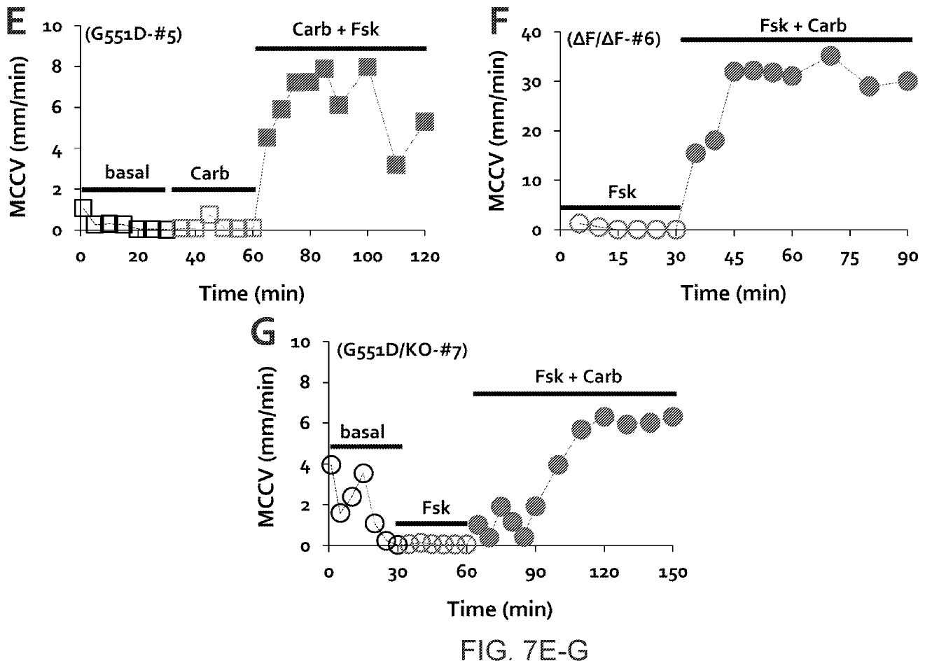

Combined agonists stimulate epithelial surface anion secretion and inhibit Na⁺ absorption. The surface epithelia also modify ASL. FIG. 4A is a cartoon of the main ion flows controlling ASL depth: anion-mediated fluid secretion increases, and Na⁺-mediated fluid absorption decreases ASL depth. It was hypothesized that the combined agonists increase ASL depth and thus MCCV by stimulating secretion and inhibiting absorption (see also FIG. 6). FIG. 4B shows our best example of an Isc trace from pig tracheal mucosa stimulated with forskolin followed by carbachol. Forskolin caused a sustained Isc increase with no measurable change in conductance. When 0.3 μM carbachol was then added, it induced a transient Isc increase (anion secretion) followed by slow decreases in Isc and conductance, with conductance reduced to 84% of the pre- and immediate post-forskolin value after ˜30 min. The ENaC inhibitor benzamil (Bz) did not cause further changes in Isc or conductance, suggesting that carbachol completely inhibited ENaC-dependent Na⁺ absorption. At this point the epithelium is secreting anions, indicated by steep drops in Isc and conductance produced by the two anion channel inhibitors, BPO-27 and niflumic acid. With no counterbalancing absorption ASL depth is predicted to increase (dotted gold line in FIG. 6A) unless MCCV increases. Our evidence shows that MCCV does increase.

FIG. 4C-F shows summary plots of ΔIsc as a function of time and stimulation. Each panel shows responses to 10 μM forskolin or 0.3 μM carbachol for the first 30 min and then the combined agonists for the next 30 min for WT pigs (FIG. 4C, D) and WT ferrets (FIG. 4E, F). Forskolin increased ΔIsc as expected for both species, but when carbachol was added the ΔIsc diminished slowly (FIG. 4C, E). Our interpretation of Isc in the forskolin+carbachol condition is that forskolin mainly increased Isc by stimulating anion secretion while carbachol largely decreased Isc by inhibiting Na+ absorption. Inhibiting Na+ absorption would increase net fluid accumulation on the surface. When carbachol is added first, the Alsc decreased directly or after a transient increase (FIG. 4D, F). In both cases the subsequent ΔIsc increase to carbachol+forskolin is smaller than to forskolin alone, because of their opposite effects on Isc but additive effects on ASL depth.

Agonist stimulation of human ciliary beat frequency (CBF). CBF is known to increase in

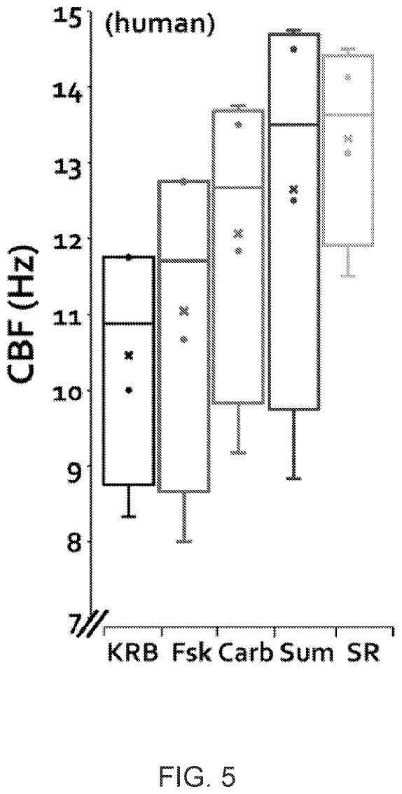

response to elevations of either [Ca²⁺]ᵢ (34) or [cAMP]ᵢ (35). It was tested to see if CBF might display synergistic increases to the combined agonists. CBF (in Hz) of unstimulated human nasal cells in KRB (Krebs buffer solution) was 6.79±1.69 at 25° C. and 10.46±0.95 at 37° C. (4 subjects, P=0.01) (see “Methods” section). As shown in FIG. 5, neither agonist increased CBF significantly, but when combined, their additive effects produced a 27.2% increase to 13.31±0.77 Hz at 37° C. This was a significant increase compared to unstimulated CBF (n=4, P<0.05), but not to the arithmetic sum of ACBF to the two agonists: combined agonists: 2.85±0.76, and arithmetic sum: 2.19±0.66 (n=4, P=0.47). Thus, while increases in CBF will contribute to increases in MCCV, they are unlikely to account for the synergistic increase in MCCV seen with the combined agonists (see “Discussion” section).

### Discussion

Main findings. There were six main findings. (1) Combined agonists produced synergistic increase of MCCV in transgenicferrets with CF to 19.95±4.12 mm/min, which is ˜55% of MCCV in WT ferrets tested in similar conditions. (2) Pigs also showed synergistic increases in MCCV, so the effect is not species-specific. (3) Little or no airway narrowing was produced by the combined agonists in WT ferrets, WT pigs, and transgenicferrets with CF. As for mechanisms: (4) glandular mucus secretion increased synergistically in in pigs, ferrets, and CF ferrets; (5) anion secretion by surface epithelia increased; (6) Na⁺ absorption by surface epithelia decreased; and (7) CBF increased. The magnitude of synergistic MCCV increases were multiple-fold higher than to either agonist alone or to their summed responses and were close to maximal values reported in vivo. In anesthetized ferrets, basal MCCV in vivo was 18.2±1.0 mm/min, and was increased to 32.0±3.8 mm/min with maximal anticholinesterase treatment (36). In anesthetized pigs, averaged basal MCCV in vivo was ˜7 mm/min, and averaged maximal MCCV was ˜12 mm/min (37). If synergy also occurs in vivo, it should help mobilize mucus in certain obstructive airway diseases.

The most noteworthy result was the synergistic increase in MCCV of CF ferrets. This has therapeutic implications, and is intriguing mechanistically because in CF ferret tracheas forskolin alone did not increase MCCV (FIG. 1A) or stimulate gland mucus secretion (FIG. 3H). (Using a different synergy paradigm, human submucosal gland secretion was lost in airways from subjects with CF (22)).

Therefore, the combined agonists used in the present study must be activating a CFTR-independent anion secretion pathway that is refractory to forskolin alone (see below).

Strategies to increase mucus clearance. Strategies to increase mucus clearance are mainstays of cystic fibrosis treatment but are only modestly effective (6,8-10). Pulmozyme (recombinant human DNase), hypertonic saline, and mannitol all improve mucus clearance in CF, while inhalation of bicarbonate or tromethamine improved CF sputum rheology (38).

Long before Pulmozyme or hypertonic saline treatments, numerous studies documented that β-adrenergic (cAMP) agonists increased MCC (13,39). Indeed β-adrenergic agonists, considered as bronchodilators, are now used ubiquitously for treating obstructive diseases. However, the doses needed to stimulate increased MCC are higher than those that reliably produce bronchodilation (39), and so it is not clear to what extent the doses presently used are increasing MCC. Unlike β-adrenergic agents, cholinergic (Ca²⁺) agents cause bronchial constriction, which is the basis for the methacholine challenge test (40), although increased mucus transport in humans by cholinergic stimulation has been reported (41). Cholinergic agents also stimulate mucus secretion, and it is widely held that mucus over-production contributes to muco-obstructive disease (42,43). Thus, it is not surprising that no one has previously advocated a therapeutic use for inhaling an agent that stimulates mucus secretion and causes bronchoconstriction. Indeed, anti-cholinergic agents are used as treatments for COPD, with modest effectiveness apparently resulting primarily from increased bronchodilation (44). Thus, our finding that a combination of forskolin (or a β-adrenergic, formoterol) and a low-dose cholinergic markedly increased MCCV was unexpected.

Our hypothesis is that the combined agonists increase MCCV mainly because they increase ASL volume via three processes: synergistic increases in gland mucus secretion, increased fluid secretion and decreased absorption by surface epithelia (FIG. 6). The combined agonists produced only modest, additive increases in CBF measured in Krebs solution. It is possible that larger increases in CBF depend on increases in ASL volume, which occurred in the MCCV experiments but not in the CBF experiments. CBF increases have been observed in response to cholinergic stimulus using micro optical coherence tomography (μOCT) to visualize transport in intact tracheas (45,46). Importantly, all of this occurs in the absence of airway narrowing.

The concept that MCCV will be faster if ASL depth is increased is supported by studies of patients with pseudohypoaldosteronism (PHA), where loss of function mutations in ENaC subunits eliminate Na+ absorption from the airway surface, which more than doubles the volume of ASL and causes a fourfold increase in 0-20 min clearance rates of inhaled tracer from the lungs (47). Previously, it was demonstrated that agonist-induced MCCV in ferrets was ˜doubled when ENaC was inhibited (16). In those experiments (16), stimulation with either forskolin or carbachol in the presence of ENAC inhibition increased MCCV to values similar to those seen with the combined agonists, providing additional evidence that synergistic MCCV results, in part, from ENaC inhibition. The idea that increased ASL provides faster clearance also underlies the logic of using β-agonists (39) and hypertonic saline (8,9) to increase clearance. It is also supported by studies of ex vivo pig tracheas, where stimulating secretion increased MCCV, blocking secretion slowed MCCV, and blocking absorption increased MCCV of tracheas after secretion had been blocked (20).

Potential molecular and cellular mechanisms. Molecular and cellular mechanisms responsible for synergistic MCCV by β-adrenergic and cholinergic agonists were not addressed in this study, but given our evidence that inhibition of ENAC contributes, prior works on molecular mechanisms of ENAC inhibition are relevant. A common theme is the role of elevated [Ca²⁺]ᵢ (48-50), which can be achieved with a wide range of agonists, including ATP, UTP, histamine, thapsigargin, and bradykinin (51). Cholinergic agonists increase [Ca²⁺]ᵢ; other mechanisms include increasing extracellular antiproteases (27,52); and other ENaC inhibitors (25,53) by stimulating secretions from airway glands and surface epithelia.

Synergistic increases of MCCV and glandular secretion were observed in CF ferrets. Therefore, mechanisms that bypass CFTR must be involved in part. Intracellular crosstalk between cAMP and Ca²⁺ signaling pathways via inositol 1,4,5-triphosphate receptor-binding protein release with IP3 (IRBIT) has been shown to mediate synergy in salivary gland and pancreatic ducts (54). Synergistic secretion by lacrimal glands in response to cAMP and cholinergic agonists was partly due to inhibition of p44/p42 mitogen-activated protein kinase (MAPK) by the cAMP agonist (55). A previous study (50) demonstrated that synergistic fluid secretion by cAMP+Ca²⁺ agonists could result from Ca²⁺ release by a cAMP-dependent Ca²⁺ release mechanism in addition to a Ca²⁺ agonist in isolated serous cells from human nasal and WT & CFTR−/−pig tracheal glands. Another study in HEK 293 cells (49), however, has shown that a cAMP agonist, such as parathyroid hormone or isoproterenol, did not increase [Ca²⁺]ᵢ by itself, but when combined with carbachol, a cAMP agonist potentiated carbachol-induced Ca²⁺ release by unmasking a discrete Ca²⁺ pool in ER. Discrepancies in previous reports might arise in part from using different cell or organ preparations and in part from using different measurement parameters, e.g., [Ca²⁺]ᵢ versus [HCO₃⁻]ᵢ ([pH]ᵢ). Our earlier studies (22,24) have shown that there are CFTR-dependent and-independent paths in synergistic glandular mucus secretions, depending on the doses of β-adrenergic and cholinergic agonists.

Potential therapeutic relevance for muco-obstructive disease. Procedures to enhance mucociliary clearance are needed for people with muco-obstructive airway disease, including substantial numbers of people with CF (11,56). Because β-agonists and methacholine are used routinely (the latter to test for hyperactive airways), little should stand in the way of testing them in combination except that it seems counterintuitive. Our ex vivo data show this combination is effective in speeding mucus clearance without inducing airway narrowing, even in CF animals (FIG. 2), which have airway muscles with increased sensitivity to cholinergic agonists (57). Our results with CF airways are consistent with an earlier study where greatly reduced bronchoconstriction was observed when a β-adrenergic agonist was administered prior to methacholine in CF children (58).

It remains to be seen if this combination is safe in individuals with hyperactive airways. If results do warrant further testing in people with CF, it will be important to start early with healthier airways, because the trend observed with β-agonist improvement of MCC was that healthier airways showed more benefit than diseased airways (39).

### Materials and Methods

Airway tissue procurement. CF ferret tissues. Seven transgenic CF ferret tracheas (five CFTR^{G551D/G551D}, one CFTR^{ΔF508/ΔF508}, one CFTR^{G551D/KO}) were used for MCC assays. These ferrets were raised on the CFTR modulator VX770. Dosing was stopped at least 3 weeks prior to euthanasia; no residual drug effect is expected or was observed (zero response to forskolin). Two CFTRKO/KO ferret tracheas were used for the tracheal, single gland mucus secretion rate assay. Two CF ferret tracheas (one CFTR^{ΔF508/ΔF508} and one CFTR^{G551D/ΔF508}) ferret trachea were used for tracheal smooth muscle contraction assay. All isolated CF ferret tracheal trims (2-3 cm in length) were placed in DMEM culture medium immediately after euthanasia and shipped from the University of Iowa via overnight priority express.

Pig tissues. Newborn WT piglet tracheas (2-5 days old) were directly obtained at the swine facility of UC Davis or from the laboratory of David Stoltz, University of Iowa, via overnight priority express. Postmortem (<1 h) tracheas from young adult Yorkshire pigs (30-50 kg) and 5-12 months old M. putorius ferrets were from animal facilities at Stanford and Gilroy/CA. All methods using animal tracheae were carried out in accordance with relevant guidelines and regulations of Stanford University and animal protocols were approved (Stanford IACUC protocol#: 10,048). Piglet tracheas were shipped in DMEM cell culture medium, other animal tissues were transported to the laboratory in cold PhysioSol™ solution (Hospira, IL/USA) and then transferred to icecold Krebs Ringer bicarbonate (KRB) buffer gassed with 95% O2 and 5% CO2 and kept at 4° C. until use. The KRB buffer contained (in mM): 115 NaCl, 25 NaHCO3, 2.4 K2HPO4, 0.4 KH2PO4, 1.2 MgCl2, 1.2 CaCl2, 10 glucose, and 1.0 μM indomethacin, adjusted to pH 7.2 and ˜290 mOsm at room temperature.

Human tissues. Human nasal mucosal tissues were obtained from nasal biopsies during endoscopic sinus surgeries at Yonsei University Hospital. All methods using human tissues were carried out in accordance with relevant guidelines and regulations of Yonsei University, Seoul, South Korea. All experimental protocols were approved by Yonsei University and informed consent was obtained from all participants prior to the study (Yonsei University-IRB protocol#: 4-2016-1153).

MCCV measurement. Details are in the previous reports (16,59,60). Briefly, each whole length ferret or piglet trachea, or a CF tracheal trim, was cut open along the mid-dorsal line and mounted mucosal side up onto a Sylgard elastomer platform. For pig experiments, tracheas from newborn piglets were used because tracheas from adult pigs that had been subjected to acute experiments, our source for submucosal gland experiment, had erratic MCC velocities due to epithelial damage secondary to intubation. The prepared trachea was placed into a sealed, humidified chamber bubbled continuously with gas (95%/5%-O₂/CO₂) with the serosal surface bathed with KRB buffer±drugs. For the initial 30 min stabilization period, the tissue was submerged in the bath as the temperature was gradually increased to 37° C. Then excess apical solution was drained, and tissue was incubated for an additional 10 min before starting baseline measurements of MCCV. Drugs were added by bath replacement with pre-warmed bath+drug(s). Summary MCCV data are reported as a single number and based on the averaged MCCV during the last 20 min of the treatment period unless stated otherwise.

Electrophysiology. Intact ferret tracheal trims (˜0.5×1.0 cm²) or pig mucosal preparations dissected from cartilage were mounted in EasyMount Ussing chambers (Physiologic Instruments, CA, USA) with exposed surface areas of 0.45 cm², bathed in KRB buffer at 37° C., and gassed with 95% O²/5% CO². Transepithelial shortcircuit current (Isc) was obtained and displayed with a VCC-600 voltage clamp (Physiologic Instruments, CA/USA), and a PowerLab Chart4 software (V. 4.1.2,https://adinstruments.com, ADInstruments, CO/USA). Total tissue conductance was calculated by applying Ohm's law to the Isc deflection resulting from a 1 mV pulse across the tissues every 20 s during the experiment. Averaged responses were reported for the last 20 min of each measurement period unless stated otherwise.

Optical measurement of glandular mucus secretion rate. Details are in the previous report (28). Intact ferret tracheal trim (˜1.5 cm²) or pig tracheal mucosa dissected from the underlying cartilage in cold Krebs Ringer bicarbonate buffer was mounted mucosal side up in a 35 mm Petri dish lined with pliable silicone so that the glands were bathed in KRB buffer while the surface was dried and covered with water-saturated mineral oil. The appearance of “mucus bubbles” within the oil layer was visualized by oblique illumination and digital images were captured with the macro lens of a Nikon digital camera. Stored images were analyzed by direct measurement or with ImageJ software (V. 1.50i, https://imagej.nih.gov/ij/, NIH, MD/USA). Rates for the indicated drugs were calculated for 5 min intervals based on averaging sustained T10-30 secretion rates by 10 mM forskolin or 0.3 mM carbachol alone or T5-30 by the combined agonists to include bubbles to be merged rapidly caused the combined agonists.

Ciliary beat frequency measurement. Ciliary beat frequency was measured using human nasal mucosa in the lab where ferret and pig tracheal mucosa was not readily accessible. Human nasal mucosa from endoscopic nasal biopsies was further dissected under a microscope and placed in a chamber controlled for temperature and pH control. Perfused Krebs bicarbonate buffer was maintained at 37° C. and pH 7.4. Cilia were visualized with a Zeiss microscope equipped with 40× or 60× objectives (Munich, Germany) using differential interference contrast (DIC) optics. Images were viewed live and were captured automatically at 2,000 fps with a high frame-rate digital camera (optiMOS and NIS-Elements microscope imaging software (Nikon, Japan)) and converted to TIFF images. Images were obtained for 10 s at each condition and experiments were performed in the following sequence: (1) unstimulated CBF at room temperature; (2) unstimulated at 37° C.; (3) CBF at 37° C. with 0.3 μM carbachol; (4) wash for 10 min; (5) CBF with 10 μM forskolin; and (6) CBF with 0.3 μM carbachol added to the forskolin. Each condition was maintained for at least 10 min. Note that this paradigm differs from the synergy paradigms used for measuring MCCV and gland mucus secretion rates in that the exposure to agonists was ≥10 min instead of 30 min, and it omits the condition in which forskolin was added in addition to carbachol. All recordings at each condition were made at three different areas of the epithelia, and the analyzed CBF was averaged for each experiment. To analyze captured images and calculate CBF, an in-house coding with a MATLAB software (MA, USA) was used.

Airway smooth muscle contraction measurement. Two methods were used to measure tracheal smooth muscle contraction. One is designed to measure airway narrowing using thin sliced tracheal rings. Piglet or ferret tracheal ring preparations of ˜2 mm were submerged and securely pinned on a Sylgard-lined Petri dish filled with KRB solution at 37° C. and pH 7.4. Digital images of tracheal ring contractions in response to agonists for 1-10 min intervals were recorded with a Nikon digital camera and the inner lumen surface area of the tracheal ring was calculated using ImageJ (NIH, MD/USA). The other method is using a force transducer. One end of an isolated ferret trachealis muscle bundle was secured in a Sylgard-lined Petri dish filled with KRB solution and the other end was attached by 26-gauge wire to a previously calibrated strain gauge (series 400A force transducer system, Cambridge Technology, MA/USA). Tension responses to increasing carbachol doses±10 μM forskolin were obtained and displayed with PowerLab Chart4 software (ADInstruments, CO/USA).

Reagents. Chemicals were purchased from Sigma-Aldrich (St. Louis, MO/USA), Calbiochem (Billerica, MA/USA), and Alomone labs (Jerusalem, Israel). BPO-27 was a generous gift from Alan Verkman, UCSF. Forskolin, benzamil, BPO-27, niflumic acid, formoterol fumarate were dissolved in dimethyl sulfoxide (DMSO) and carbachol was dissolved in sterile double distilled water and indomethacin was dissolved in absolute ethanol. Solutions were made fresh or maintained at −20° C. as aliquots of stock concentration. All chemicals were diluted 1:1000 with bath KRB solution (except indomethacin, 1:10,000) immediately before use at the concentrations indicated.

Statistics. Data is presented as mean±S.E.M. unless otherwise indicated. To compare means of different treatment groups, either Student's paired and unpaired t-test or Mann-Whitney U test was used.

### Example 2

### Effects of Sequential and Simultaneous Administration of Formoterol and Methacholine on Mucociliary Clearance Velocity

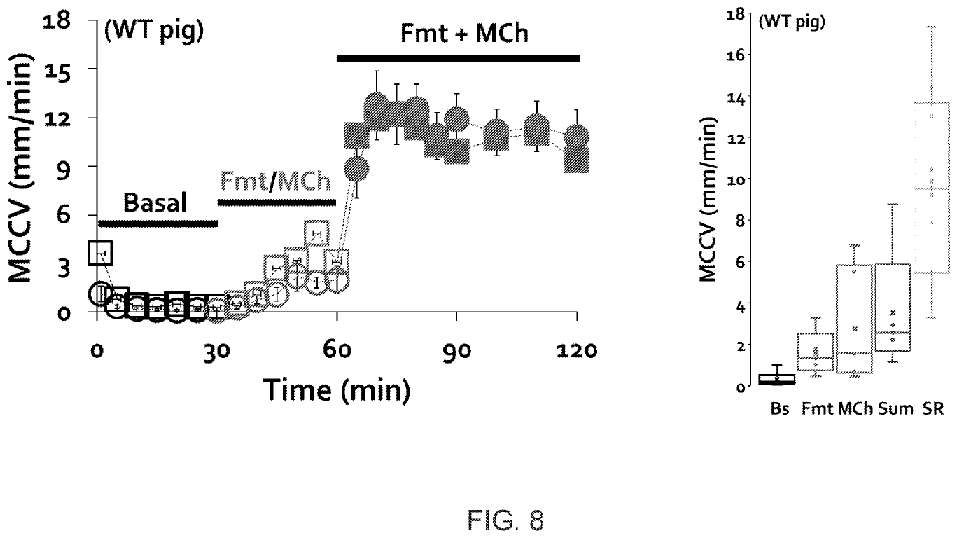

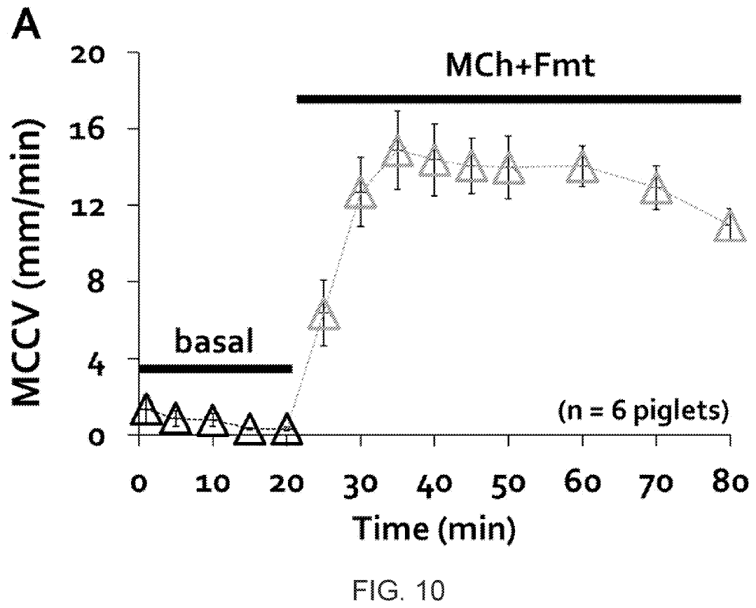

To determine if methacholine and formoterol could also induce a synergistic increase in MCCV, piglet trachcas were treated with 0.3 μM methacholine followed by 10 μM formoterol or 10 μM formoterol followed by 0.3 μM methacholine. MCCV (all values are in mm/min) was then measured for 90-120 min and plotted as MCCV vs time and agonist(s) in FIG. 8. MCCV declined to near zero in the first 30 min without stimulation (FIG. 8). Formoterol produced no increase in MCCV and methacholine produced only a small increase. However, when the agonists were then combined in either order, they produced large, sustained increases in MCCV to ˜12 mm/min. This synergistic increase in MCCV was observed when methacholine and formoterol were administered simultaneously (FIG. 10)

### Effects of Sequential and Simultaneous Administration of Formoterol and Methacholine on Airway Smooth Muscle Contraction

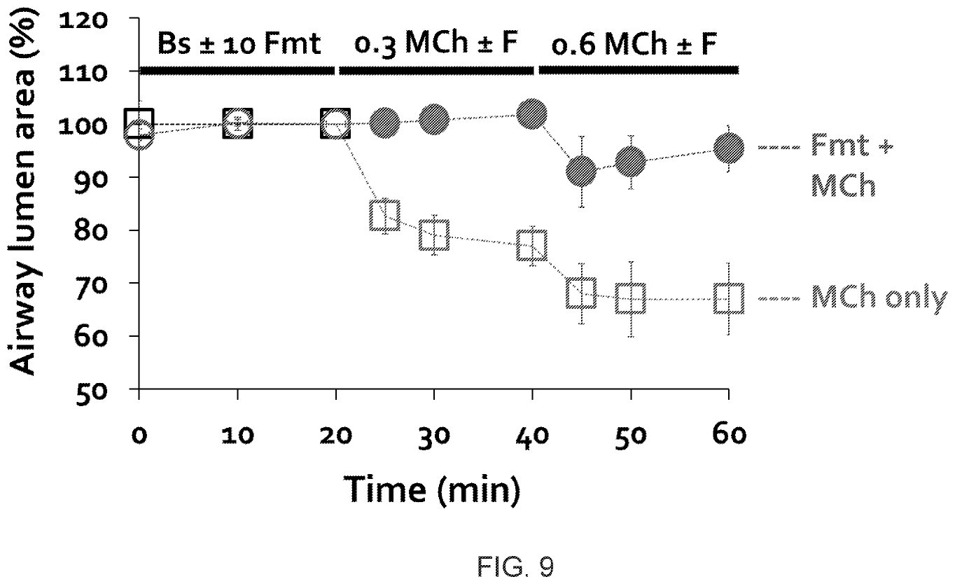

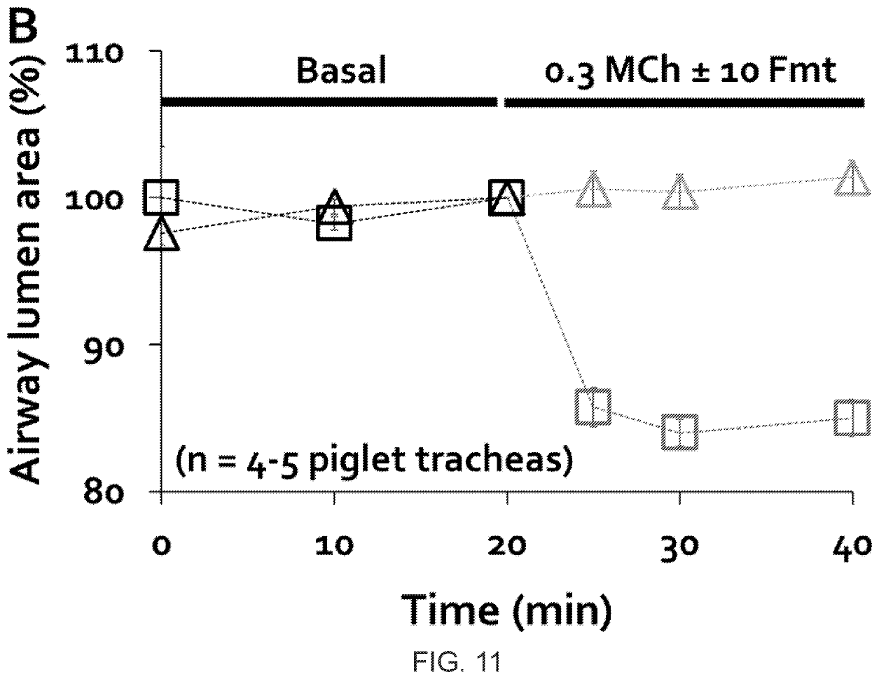

To determine if formoterol could also suppress cholinergic agonist induced airway smooth muscle contraction, airway smooth muscle responses to methacholine (0.3 or 0.6 μM)±10 mM formoterol was measured by measuring lumen area. Lumen area, imaged in thin sliced piglet tracheal rings, displayed a sustained 20-40% reduction with exposure to 0.3 or 0.6 mM methacholine, but when methacholine was preceded by formoterol, it induced only a transient decrease of 5-10% or less (FIG. 9). The protection against methacholine induced airway smooth muscle contraction was also observed when methacholine and formoterol were administered simultaneously (FIG. 11)

### Effects on Mucus Transport at the Epithelial Level in an In Vitro Cell Culture Model

Human nasal epithelial cells (HNECs) were obtained from patient with CF (F508 del Homozygote) and healthy WT control following an established standard operating procedure. The cells were then dissociated, seeded onto collagen-coated, 0.4-μm pore polyester membrane inserts (Corning Inc.) and expanded with Pneumacult Ex-Plus media (StemCell Technologies) and added to both the basal and apical chambers. This was followed by removing the apical medium to create an air-liquid interface (ALI) and replacing the basal medium with Pneumacult ALI media (StemCell Technologies). Once HNEC ALI cultures were mature as evidenced by presence of active cilia, CF cells were treated for 48 hrs with either DMSO control or the combination Elexacaftor (3 μM)-Tezacaftor (3 μM)-Ivacaftor (10 μM) (ETI) After 48 hrs the cells were treated with either DMSO control, forskolin, carbachol or forskolin+carbachol (SP) and the inserts were cut from their support, placed on a concave well slide filled with media so that only the basal side was exposed to media, and 20 μL of a 0.1% suspension of 2 μm fluorescent polystyrene beads (Thermo Scientific R0200) were added to the apical surface.

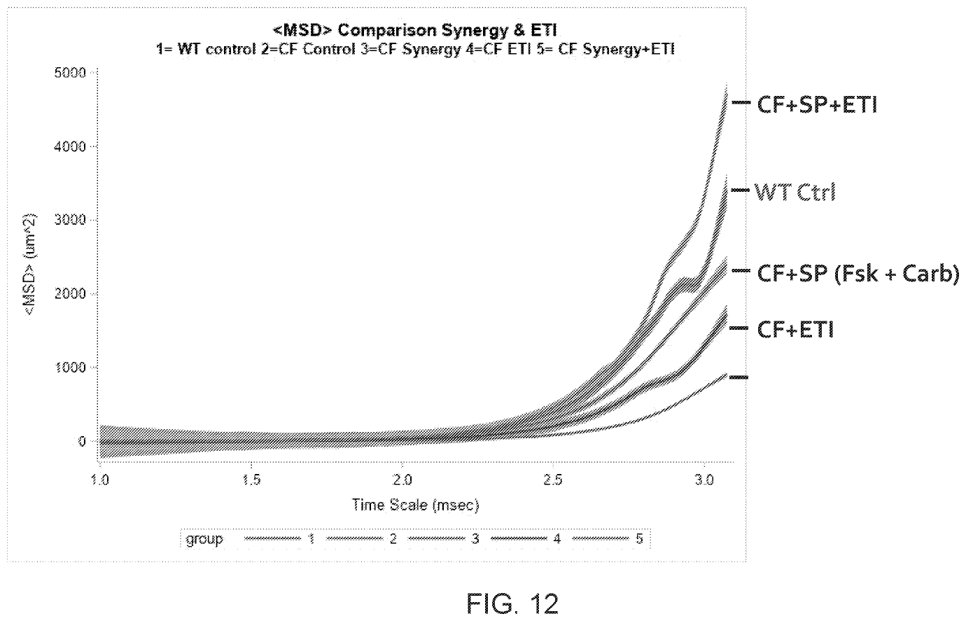

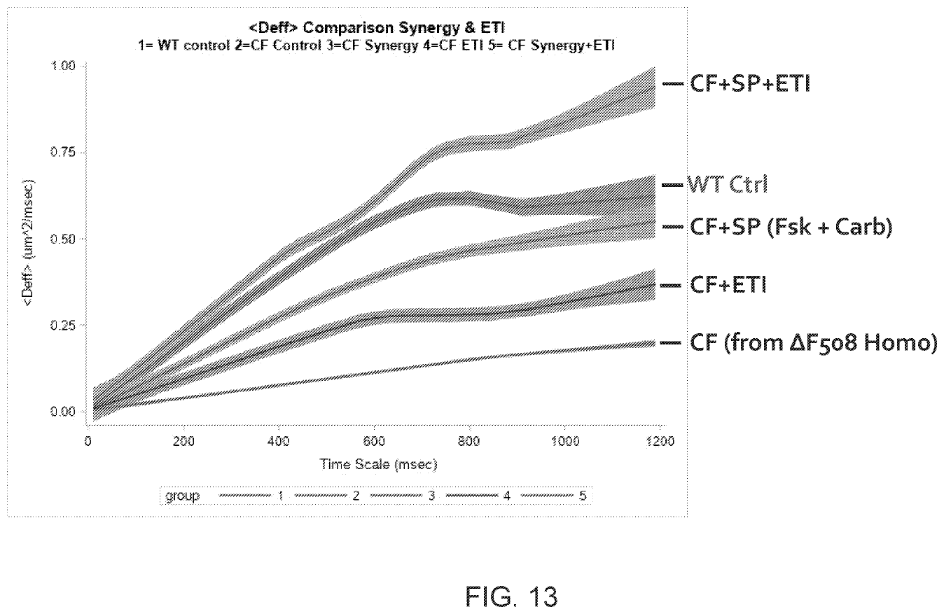

The slide was then placed on a custom-built system that includes a heated stage at 37° C. and imaged from above with a digital microscope fitted with a high-speed camera (Keyence Inc., Elmwood Pk, NJ). The images were acquired at a frame rate of 1000 fps and exported to Image J to analyze particle movement with the MTrackJ plug-in (v. 1.5.1). Wild type cells served as a reference control. The extracted frame-by-frame coordinates were then used to estimate individual particle distance travelled. The coordinates and time lag between frames were used to define time scales (τ) following multiple particle transport (MPT) methodology and then determine the mean squared displacement (MSD) as the particle square displacement for all possible time lags. The effective diffusivity (D_{eff}) was then calculated from the MSD and time scales extracted. The MSD and D_{eff} data generated for all particles tracked were analyzed by Loess smoothed regression to generate ensemble plots of MSD and D_{eff} vs time scales with 95% confidence intervals to allow for comparisons between treatment conditions (FIGS. 12 and 13).

### Example 3

Combined Agonists when Administered by Inhalation Greatly Improve MCC in Sheep with Induced CF Airway Disease.

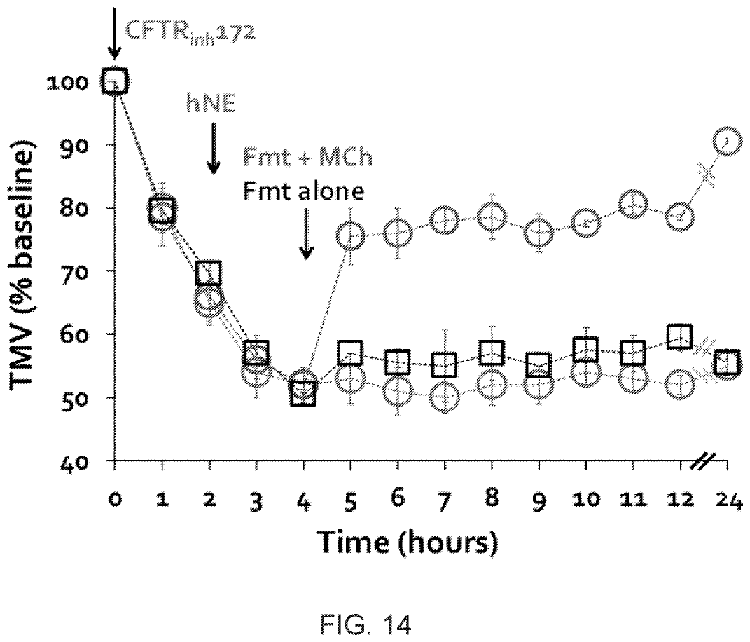

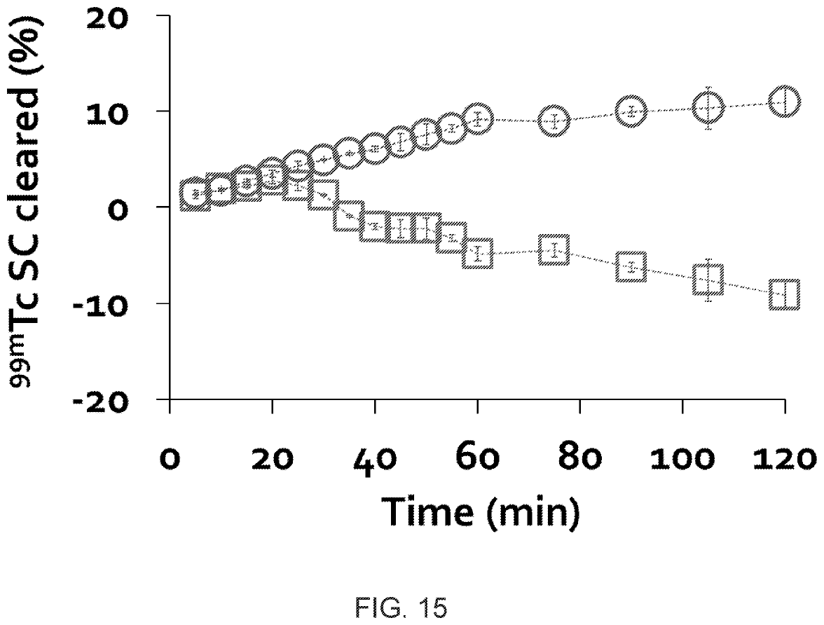

To determine if synergistic increases of MCCV observed in ex vivo and in vivo experiments above could be produced in vivo in a CF animal model studies were conducted with adult sheep. In this well-established model of CF (Abraham, W. Pulm Pharm Ther 2008; Kim et al; Am J Respir Crit Care Med 2020, 201:313-324), CF-like conditions are created in the airways of adult sheep by the aerosolization of CFTRinh172 followed by human neutrophil elastase. As a large animal model, it provides the opportunity to evaluate for effects on a tracheobronchial tree with similar characteristics to humans with CF, as well as almost directly translating inhalational dosing considerations. Briefly, adult sheep are placed in an upright position in a specialized body harness and under local anesthesia the animals are nasally intubated with a standard endotracheal (ET) tube. Tracheal mucus velocity (TMV), the in vivo equivalent of MCCV, is measured by a fluoroscopic technique. Five to seven radiopaque Teflon/bismuth trioxide disks (1 mm in diameter, 0.8-mm in thickness, and 1.8 mg in weight) are insufflated into the mid-portion of the animal's trachea. Once the disks are delivered into the trachea, the cephalad-axial velocity of each individual disk is recorded on videotape from a portable image intensifier unit in-line with the fluoroscope. The mean value of all the disk velocities is calculated for a given time point, with new disks insufflated pat subsequent time points. After baseline values are determined, animals receive by aerosolization 10 mg of CFTRinh172 and TMV measurements are repeated hourly for the following 2 hours. Then the animals receive by aerosolization 1190 mU of human Neutrophil Elastase (hNE) and TMV measurements are repeated hourly for the following 2 hours. This induces a profound and persistent impairment in TMV, typical of CF. Once the CF conditions were established the animals received by inhalation either a vehicle control (n=4), 20 μg of Formoterol (n=2), or a combination of 20 μg of Formoterol plus 12 μg of Methacholine (n=2). Methacholine alone was not tested as this will induce severe bronchospasm and compromise the life of the animal. After test agent administration by inhalation, the TMV was measured hourly for 12 hours and at 24 hours. As shown in FIG. 14 after suppression of TMV by CFTRinh172+hNE, Formoterol alone barely had any effect on TMV compared to the control, and the synergy agonists increased TMV to ˜80% of the pre-suppressed baseline. Importantly, the effect was sustained for 24 hours after a single dose administration. Since the TMV studies only assess MCC in the trachea and our ultimate goal is to resolve mucus accumulation in the lungs, the TMV studies were complemented with whole lung clearance studies. To accomplish this, the same CF sheep model was used but assessed whole lung MCC by radionuclide clearance technique. For this, after the administration of CFTRinh172 and hNE, 99mTc-sulphur colloid was administered by inhalation and followed by inhalation of either a vehicle control or the combination of 20 μg of Formoterol plus 12 μg of Methacholine. Then the radiotracer activity in the lungs was monitored with a gamma camera over a 2-hour period as described before (Abraham, Pulm Pharmacol Ther 2008, 21, 743-75). As shown in FIG. 15, under control conditions (n=2) there was retention of the radiotracer over the 2-hour observation period, with an artifactual appearance of an increase in signal, as opposed to the synergy treated animals (n=2) were radiotracer activity decreased by 11%. Of particular note is that no untoward effects were observed in the sheep exposed to the synergy combination.

Combined Agonists Administered by Inhalation as a Single Dose are Well Tolerated by Healthy Human Volunteers and Patients with CF.

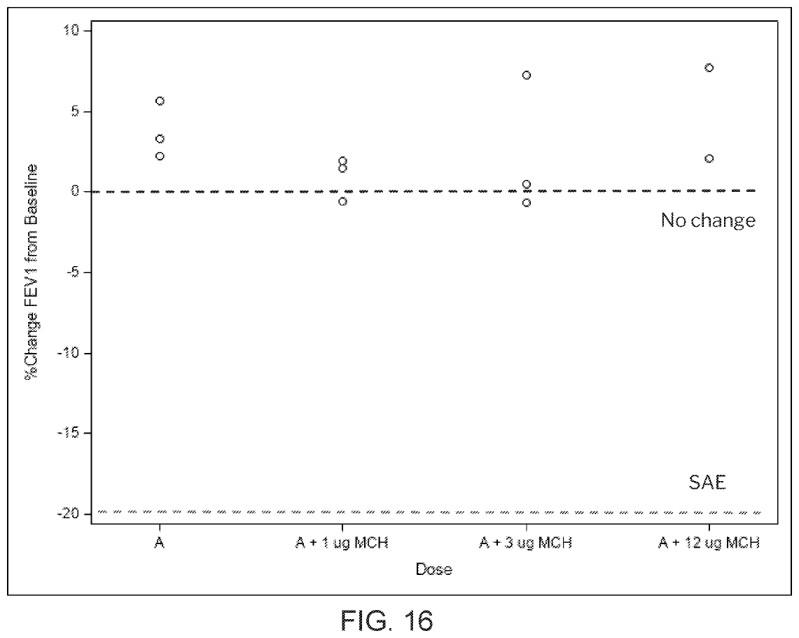

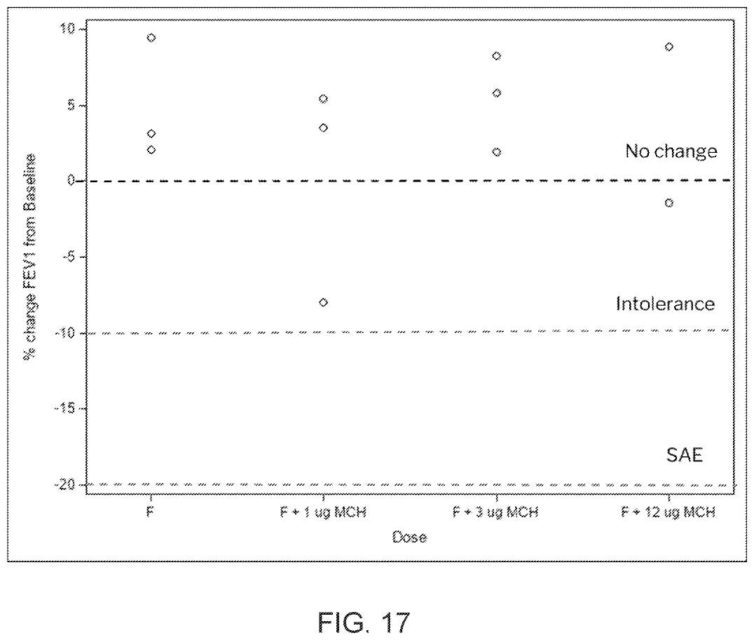

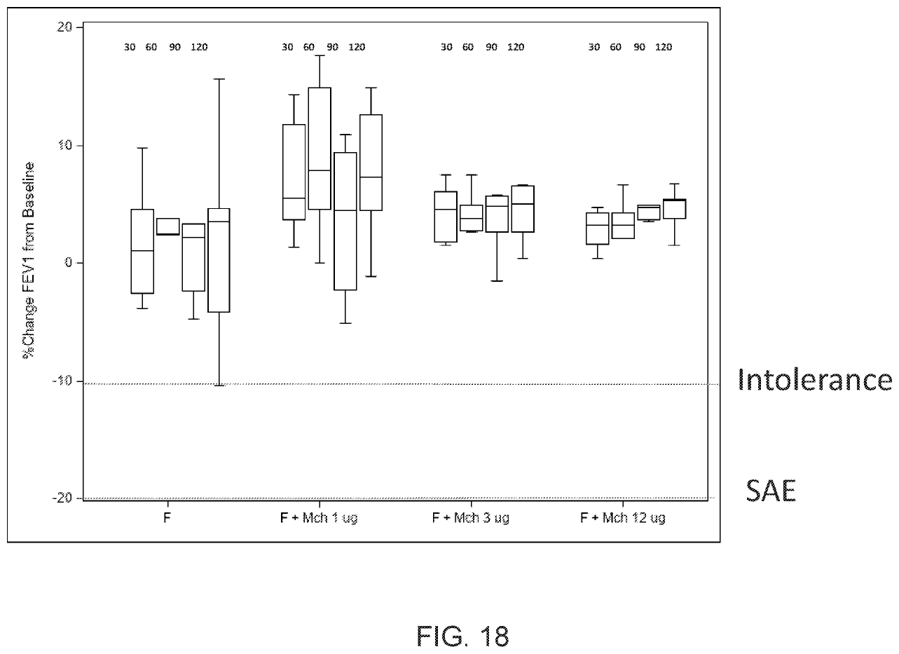

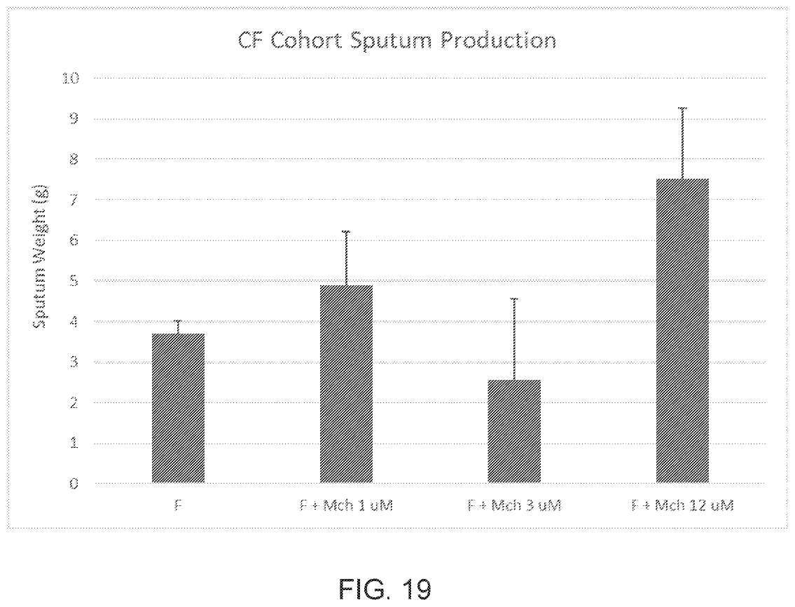

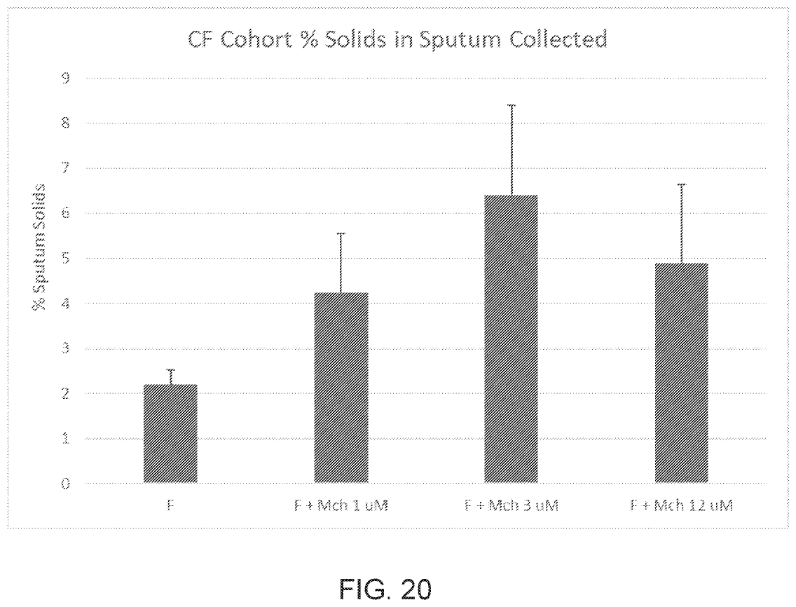

Encouraged by the experimental results it was desired to evaluate if a combination of a β-adrenergic and a cholinergic agonist delivered by inhalation is tolerated by humans, as this will establish the basis to proceed to full development of a therapeutic for clinical application. A series of 3 dose escalation, single administration studies with human volunteers and patients with CF were conducted. For these studies, we made use of available clinical grade drugs. In the first study, it was desired to test the effects with the short-acting β-adrenergic agent albuterol and in combination with methacholine at 0, 1, 3, or 12 μg in dosing cohorts of 3 healthy volunteers each. Overall, all doses tested were well tolerated and no untoward effects were noted, meeting a pre-defined safety endpoint of no individuals experiencing a drop in Forced Expiratory Volume in 1 second (FEV1, a standard measure of airway obstruction) of 20 points or larger (FIG. 16). This study was followed by a similar study but this time using the long-acting β-adrenergic agent formoterol, with which most of our experimental work was conducted. The study was similar to the first study, with human volunteers in cohorts of 3 subjects receiving by inhalation Formoterol and in combination with methacholine at 0, 1, 3, or 12 μg. Again, we observed no signs of intolerance to any of the doses tested and the study met its predefined safety endpoint, this time set more stringently as a drop in FEV1 of 10 points or larger (FIG. 17). With these encouraging results, a study with patients with CF was conducted, similar to the healthy volunteer study with formoterol and Methacholine, but with the only difference being an increase in the dosing cohorts size to 6 subjects. As with the previous studies, no untoward effects were noted at any dose tested and the study also met its safety endpoint of no drop in FEV1>10 points (FIG. 18). As exploratory endpoints in the CF study, the sputum production by the subjects was evaluated, as a reflection of an effect on mucus clearance from the lungs. It was noted that an increase in sputum produced (in g, FIG. 19) that was larger at the highest dose of Methacholine, though this did not reach statistical significance likely owing to the small sample sizes for each dosing cohort (p=0.056). However, a statistically significant increase was found in the sputum solids content in the groups receiving Methacholine (p=0.024; FIG. 20) which we interpret as reflecting the mobilization of more dense material as is characteristic of the retained secretions in the CF lung.

### Example 4

Further Evidence that Inhibition of ENaC-Mediated Transport is a Component of Synergistic Increases in MCCV.

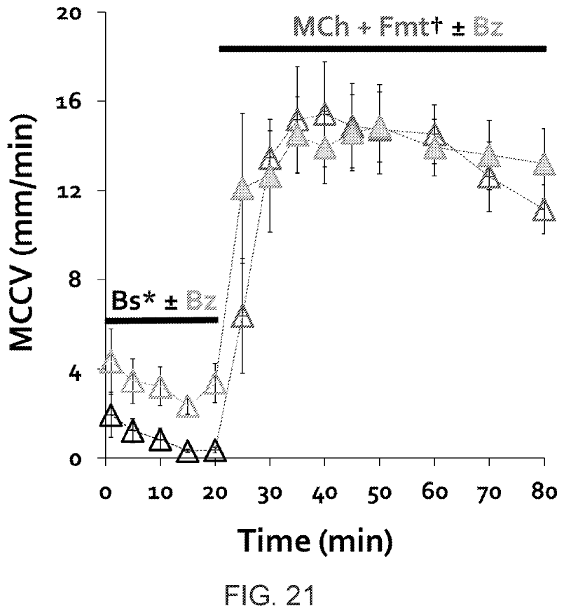

Previously it was demonstrated that stimulating ferret tracheas with forskolin or carbachol in the presence of ENAC inhibition increased MCCV values similar to those seen with the combined agonists, indicating a potential involvement of ENaC inhibition in the synergistic MCC induced by the combined agonists. It was hypothesized that the synergistic MCC induced by β-adrenergic and cholinergic agonists involved ENaC inhibition. Consistent with this hypothesis, the results of MCCV by 10 μM formoterol+0.3 μM methacholine are comparable in the presence (10 μM benzamil) or absence (0.1% DMSO as a control treatment) of ENAC inhibition (in mm/min): 14.0±1.6 with benzamil and 13.9±1.6 without benzamil (P=0.97, n=4 piglets). As shown in FIG. 21, baseline MCCV was significantly increased, almost 6 times, by ENaC inhibition: 3.0±0.7 with benzamil and 0.5±0.7 without benzamil (P=0.02, 4 piglets).

### ASL pH Can Influence Mucus Viscosity and Mucociliary Clearance.

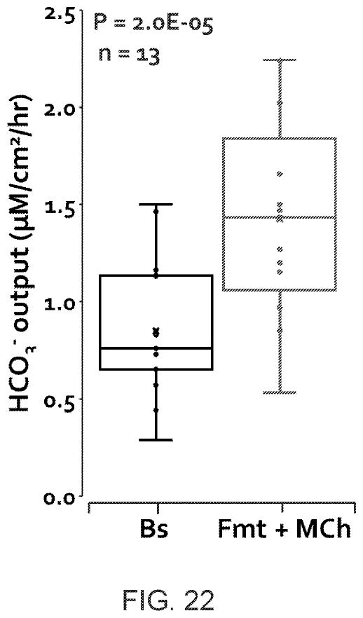

In general, viscoelasticity of mucus increases, and clearance slows when pH is lowered. It was desired to assess if combined agonists increased HCO₃⁻ secretion from airways. Pig tracheal mucosa and pH stat was used to assess HCO₃⁻ production and found that 10 μM formoterol+0.3 μM methacholine increased HCO₃⁻ secretion rates by 68% (in μM/cm2/hr): 0.76±0.11 at baseline and 1.24±0.14. (FIG. 22). Increase HCO₃⁻ secretion by the synergy agonist stimulation. Synergy agonists significantly increased the HCO₃⁻ secretion rate when compared to baseline conditions (p=2E−05, n=13 from 8 pig tracheas).

### Synergy Agonists Increased ASL Height In Ex Vivo WT Pig Tracheas.

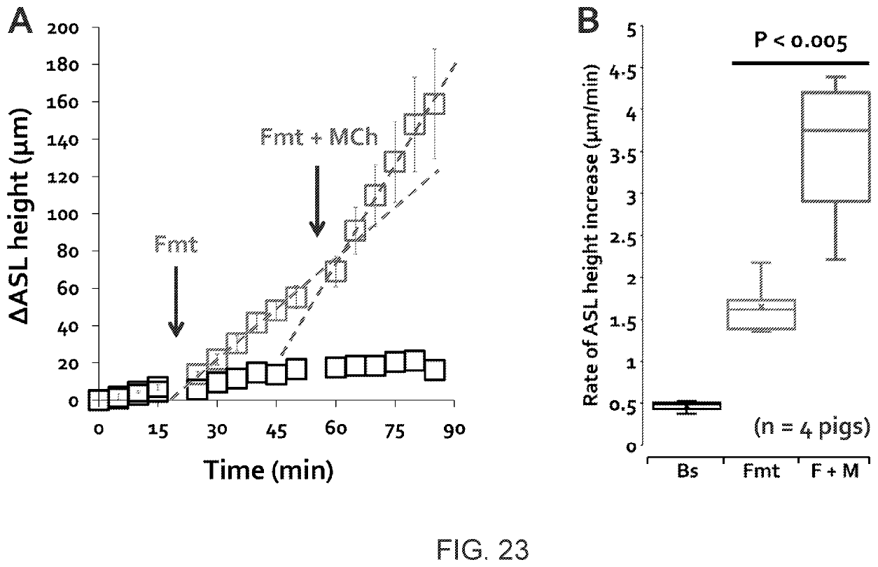

ASL heights were determined in pig tracheal preparations using synchrotron-based phase contrast imaging (PCI). The synchrotron beamline produces enough parallel spatial coherence X-ray to increase the contrast between airway lumen and ASL layer by PCI. When X-rays pass through the intact tracheal preparation, the difference in refractive index between ASL and airway lumen results in a phase shift of X-rays that causes a distinctive interference pattern shown on the charge-coupled device (CCD) detector as variations in X-ray intensity. The ASL height measurements were obtained every 5 min after the instillation of agarose beads that served as a measuring barometer: T15-baseline, T30-10 μM formoterol alone, follow by T30-0.3 μM methacholine+10 μM formoterol. As shown in FIG. 23A, both the β adrenergic alone and the combined synergy agonists (n=4 pig tracheas) increased ASL heights, while control pigs (n=2 pig tracheas) treated with vehicle (DMSO-treated) failed to increase ASL height during the same measurement period. The average rate of ASL height increase during the period of synergy drug is significantly larger (P<0.005, n=4 pigs) than that of formoterol period (in μm/min): baseline (T5-T15), 0.5; 10 μM formoterol (T30-T50), 1.7±0.1; and the synergy agonists (T65-T85), 3.6±0.4 (FIG. 23B).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

### References

1. Pezzulo, A. A. et al. Reduced airway surface pH impairs bacterial killing in the porcine cystic fibrosis lung. Nature 487, 109-113. https://doi.org/10.1038/nature11130 (2012).
2. Matsui, H. et al. Evidence for periciliary liquid layer depletion, not abnormal ion composition, in the pathogenesis of cystic fibrosis airways disease. Cell 95, 1005-1015 (1998).
3. Wine, J. J. How to live a long and healthy life with cystic fibrosis: Lessons from the CF ferret. J. Cyst. Fibros 18, 8-9. https://doi.org/10.1016/j.jcf.2018.10.005 (2019).
4. Moss, R. B. Infection, inflammation, and the downward spiral of cystic fibrosis lung disease. J. Pediatr. 154, 162-163. https://doi.org/10.1016/j.jpeds.2008.09.042 (2009).
5. Rosen, B. H. et al. Infection is not required for mucoinflammatory lung disease in CFTR-knockout ferrets. Am. J. Respir. Crit. Care Med. 197, 1308-1318. https://doi.org/10.1164/rccm.201708-1616OC (2018).
6. Fuchs, H. J. et al. Effect of aerosolized recombinant human DNase on exacerbations of respiratory symptoms and on pulmonary function in patients with cystic fibrosis; The Pulmozyme Study Group. N. Engl. J. Med. 331, 637-642. https://doi.org/10.1056/NEJM1 99409 08331 1003 (1994).
7. Frederiksen, B., Pressler, T., Hansen, A., Koch, C. & Hoiby, N. Effect of aerosolized rhDNase (Pulmozyme) on pulmonary colonization in patients with cystic fibrosis. Acta Paediatr. 95, 1070-1074. https://doi.org/10.1080/08035 25060 07524 66 (2006).
8. Robinson, M. et al. Effect of hypertonic saline, amiloride, and cough on mucociliary clearance in patients with cystic fibrosis. Am. J. Respir. Crit. Care Med. 153, 1503-1509 (1996).
9. Elkins, M. R. et al. A controlled trial of long-term inhaled hypertonic saline in patients with cystic fibrosis. N. Engl. J. Med. 354, 229-240 (2006).
10. Nevitt, S. J., Thornton, J., Murray, C. S. & Dwyer, T. Inhaled mannitol for cystic fibrosis. Cochrane Database Syst. Rev. 2, CD008649. https://doi.org/10.1002/14651 858.CD008 649.pub3 (2018).
11. Middleton, P. G. et al. Elexacaftor-tezacaftor-ivacaftor for cystic fibrosis with a single phe508del allele. N. Engl. J. Med. 381, 1809-1819. https://doi.org/10.1056/NEJMo a1908 639 (2019).
12. Van Goor, F. et al. Rescue of CF airway epithelial cell function in vitro by a CFTR potentiator, VX-770. Proc. Natl. Acad. Sci. U S A 106, 18825-18830. https://doi.org/10.1073/pnas.09047 09106 (2009).
13. Wanner, A., Salathe, M. & O'Riordan, T. G. Mucociliary clearance in the airways. Am. J. Respir. Crit. Care Med. 154, 1868-1902 (1996).
14. Button, B. et al. A periciliary brush promotes the lung health by separating the mucus layer from airway epithelia. Science 337, 937-941. https://doi.org/10.1126/science.12230 12 (2012).
15. Roy, M. G. et al. Muc5b is required for airway defence. Nature 505, 412-416. https://doi.org/10.1038/nature12807nature12807 (2014).
16. Joo, N. S., Jeong, J. H., Cho, H. J. & Wine, J. J. Marked increases in mucociliary clearance produced by synergistic secretory agonists or inhibition of the epithelial sodium channel. Sci. Rep. 6, 36806. https://doi.org/10.1038/srep3 6806 (2016).
17. Button, B., Anderson, W. H. & Boucher, R. C. Mucus hyperconcentration as a unifying aspect of the chronic bronchitic phenotype. Ann. Am. Thorac. Soc. 13(Suppl 2), S156-162. https://doi.org/10.1513/Annal sATS. 201507-455KV (2016).
18. Quinton, P. M. Cystic fibrosis: Impaired bicarbonate secretion and mucoviscidosis. Lancet 372, 415-417. https://doi.org/10.1016/S0140-6736(08) 61162-9 (2008).
19. Tang, X. X. et al. Acidic pH increases airway surface liquid viscosity in cystic fibrosis. J. Clin. Invest. 126, 879-891. https://doi.org/10.1172/JCI83 922 (2016).
20. Ballard, S. T., Trout, L., Mehta, A. & Inglis, S. K. Liquid secretion inhibitors reduce mucociliary transport in glandular airways. Am. J. Physiol. 283, L329-335 (2002).
21. Cho, H. J., Joo, N. S. & Wine, J. J. Mucus secretion from individual submucosal glands of the ferret trachea. Am. J. Physiol. 299, L124-136 (2010).
22. Choi, J. Y. et al. Synergistic airway gland mucus secretion in response to vasoactive intestinal peptide and carbachol is lost in cystic fibrosis. J. Clin. Invest. 117, 3118-3127 (2007).
23. Choi, J. Y. et al. Substance P stimulates human airway submucosal gland secretion mainly via a CFTR-dependent process. J. Clin. Invest. 119, 1189-1200. https://doi.org/10.1172/JCI37 284 (2009).
24. Joo, N. S., Cho, H. J., Khansaheb, M. & Wine, J. J. Hyposecretion of fluid from tracheal submucosal glands of CFTR-deficient pigs. J. Clin. Invest. 120, 3161-3166. https://doi.org/10.1172/JCI43 466 (2010).
25. Joo, N. S. et al. Proteomic analysis of pure human airway gland mucus reveals a large component of protective proteins. PLoS ONE 10, e0116756. https://doi.org/10.1371/journal.pone.01167 56 (2015).
26. Joo, N. S., Krouse, M. E., Choi, J. Y., Cho, H. J. & Wine, J. J. Inhibition of airway surface fluid absorption by cholinergic stimulation. Sci. Rep. 6, 20735. https://doi.org/10.1038/srep2 0735 (2016).
27. Joo, N. S., Lee, D. J., Winges, K. M., Rustagi, A. & Wine, J. J. Regulation of antiprotease and antimicrobial protein secretion by airway submucosal gland serous cells. J. Biol. Chem. 279, 38854-38860. https://doi.org/10.1074/jbc.M4070 77200 (2004).
28. Joo, N. S., Wu, J. V., Krouse, M. E., Saenz, Y. & Wine, J. J. Optical method for quantifying rates of mucus secretion from single submucosal glands. Am. J. Physiol. 281, L458-468 (2001).
29. Sun, X. et al. Disease phenotype of a ferret CFTR-knockout model of cystic fibrosis. J. Clin. Invest. 120, 3149-3160. https://doi.org/10.1172/JCI43 052 (2010).
30. Widdicombe, J. H. & Wine, J. J. Airway gland structure and function. Physiol. Rev. 95, 1241-1319. https://doi.org/10.1152/physrev.00039.2014 (2015).
31. Al-Bazzaz, F. J. Regulation of Na and Cl transport in sheep distal airways. Am. J. Physiol. 267, L193-198. https://doi.org/10.1152/ajplung.1994.267.2.L193 (1994).
32. Al-Bazzaz, F. J. & Gailey, C. Ion transport by sheep distal airways in a miniature chamber. Am. J. Physiol. 281, L1028-1034. https://doi.org/10.1152/ajplung.2001.281.4.L1028 (2001).
33. Knowles, M. et al. Bioelectric properties and ion flow across excised human bronchi. J. Appl. Physiol. Respir. Environ. Exerc. Physiol. 56, 868-877. https://doi.org/10.1152/jappl.1984.56.4.868 (1984).
34. Salathe, M., Lipson, E. J., Ivonnet, P. I. & Bookman, R. J. Muscarinic signaling in ciliated tracheal epithelial cells: Dual effects on Ca2+ and ciliary beating. Am. J. Physiol. 272, L301-310 (1997).
35. Schmid, A. et al. Real-time analysis of cAMP-mediated regulation of ciliary motility in single primary human airway epithelial cells. J. Cell Sci. 119, 4176-4186. https://doi.org/10.1242/jcs.03181 (2006).
36. Marin, M. G., Zwierzynski, D., McBride, R. K. & Culp, D. J. Effects of soman and its antidotes on tracheal mucociliary transport of ferrets. J. Appl. Physiol. 1985(66), 1390-1396. https://doi.org/10.1152/jappl.1989.66.3.1390 (1989).
37. Hoegger, M. J. et al. Assessing mucociliary transport of single particles in vivo shows variable speed and preference for the ventral trachea in newborn pigs. Proc. Natl. Acad. Sci. U S A 111, 2355-2360. https://doi.org/10.1073/pnas.13236 33111 (2014).
38. Gomez, C. C. S. et al. Safety, tolerability, and effects of sodium bicarbonate inhalation in cystic fibrosis. Clin. Drug Investig. 40, 105-117. https://doi.org/10.1007/s40261-019-00861-x (2020).
39. Bennett, W. D. Effect of beta-adrenergic agonists on mucociliary clearance. J. Allergy Clin. Immunol. 110, S291-297. https://doi.org/10.1067/mai.2002.129704 (2002).
40. Coates, A. L. et al. ERS technical standard on bronchial challenge testing: General considerations and performance of methacholinechallenge tests. Eur. Respir. J. https://doi.org/10.1183/13993 003.01526-2016 (2017).
41. Camner, P., Strandberg, K. & Philipson, K. Increased mucociliary transport by cholinergic stimulation. Arch. Environ. Health 29,220-224 (1974).
42. Reid, L. Treatment of mucus hypersecretion in human disease. Adv. Exp. Med. Biol. 89, 469-476(1977).
43. Rogers, D. F. Mucus hypersecretion in chronic obstructive pulmonary disease. Novartis Found. Symp. 234, 65-77 (2001).
44. Casaburi, R. et al. A long-term evaluation of once-daily inhaled tiotropium in chronic obstructive pulmonary disease. Eur. Respir. J. 19, 217-224. https://doi.org/10.1183/09031 936.02.00269 802 (2002).
45. Lin, V. Y. et al. Excess mucus viscosity and airway dehydration impact COPD airway clearance. Eur. Respir. J. https://doi.org/10.1183/ 13993 003. 00419-2019 (2020).
46. Liu, L. et al. Method for quantitative study of airway functional microanatomy using micro-optical coherence tomography. PLoS ONE 8, e54473. https://doi.org/10.1371/ journal.pone.00544 73 (2013).
47. Kerem, E. et al. Pulmonary epithelial sodium-channel dysfunction and excess airway liquid in pseudohypoaldosteronism. N. Engl. J. Med. 341, 156-162 (1999).
48. Gu, Y. Effects of [Ca2+]i and pH on epithelial Na+ channel activity of cultured mouse cortical collecting ducts. J. Exp. Biol. 211,3167-3173. https://doi.org/10.1242/jeb.019646 (2008).
49. Konieczny, V., Tovey, S. C., Mataragka, S., Prole, D. L. & Taylor, C. W. Cyclic AMP recruits a discrete intracellular Ca(2+) store by unmasking hypersensitive IP3 receptors. Cell Rep. 18, 711-722. https://doi.org/10.1016/j.celrep.2016.12.058 (2017).
50. Lee, R. J. & Foskett, J. K. cAMP-activated Ca2+ signaling is required for CFTR-mediated serous cell fluid secretion in porcine and human airways. J. Clin. Invest. 120, 3137-3148. https://doi.org/10.1172/JCI42 992 (2010).
51. Devor, D. C. & Pilewski, J. M. UTP inhibits Na+ absorption in wild-type and DeltaF508 CFTR-expressing human bronchial epithelia. Am. J. Physiol. 276, C827-837. https://doi.org/10.1152/ajpce ll.1999.276.4.C827 (1999).
52. Evans, T. I. et al. Glandular proteome identifies antiprotease cystatin c as a critical modulator of airway hydration and clearance. Am. J. Respir. Cell Mol. Biol. 54, 469-481. https://doi.org/10.1165/rcmb.2015-00900C (2016).
53. Garcia-Caballero, A. et al. SPLUNC1 regulates airway surface liquid volume by protecting ENaC from proteolytic cleavage. Proc. Natl. Acad. Sci. U S A 106, 11412-11417. https://doi.org/10.1073/ pnas.09036 09106 (2009).
54. Park, S. et al. Irbit mediates synergy between ca(2+) and cAMP signaling pathways during epithelial transport in mice. Gastroenterology 145, 232-241. https://doi.org/10.1053/j.gastro.2013.03.047 (2013).
55. Funaki, C., Hodges, R. R. & Dartt, D. A. Role of cAMP inhibition of p44/p42 mitogen-activated protein kinase in potentiation of protein secretion in rat lacrimal gland. Am. J. Physiol. Cell Physiol. 293, C1551-1560. https://doi.org/10.1152/ajpce11.00013.2007 (2007).
56. Veit, G. et al. Allosteric folding correction of F508del and rare CFTR mutants by elexacaftor-tezacaftor-ivacaftor (Trikafta) combination. JCI Insight https://doi.org/10.1172/jci.insight.139983 (2020).
57. Cook, D. P. et al. Cystic fibrosis transmembrane conductance regulator in sarcoplasmic reticulum of airway smooth muscle: implications for airway contractility. Am. J. Respir. Crit. Care Med. 193, 417-426. https://doi.org/10.1164/rccm.201508-15620C (2016).
58. Avital, A., Sanchez, I. & Chernick, V. Efficacy of salbutamol and ipratropium bromide in decreasing bronchial hyperreactivity in children with cystic fibrosis. Pediatr. Pulmonol. 13, 34-37. https://doi.org/10.1002/ppul.19501 30109 (1992).
59. Jeong, J. H., Joo, N. S., Hwang, P. H. & Wine, J. J. Mucociliary clearance and submucosal gland secretion in the ex vivo ferret trachea. Am. J. Physiol. 307, L83-93. https://doi.org/10.1152/ajplung.00009.2014 (2014).
60. Nam Soo Joo, Hyung-Ju Cho, Meagan Shinbashi, Jae Young Choi, Carlos E Milla, John F Engelhardt, Jeffrey J Wine Combined agonists act synergistically to increase mucociliary clearance in a cystic fibrosis airway model Sci Rep (2021)

Notwithstanding the appended claims, the disclosure set forth herein is also described by the following clauses:

1. A method of treating an individual for a muco-obstructive disorder, the method comprising: administering to the individual a β-adrenergic agonist or an adenylyl cyclase activator, in combination with a cholinergic agonist to treat the individual for the muco-obstructive disorder.

2. The method of clause 1, wherein the muco-obstructive disorder is selected from the group consisting of cystic fibrosis, primary ciliary dyskinesia, asthma, chronic obstructive pulmonary disease (COPD), idiopathic pulmonary fibrosis, and chronic bronchitis.

3. The method of clause 1, wherein the-adrenergic agonist is a β₂-adrenergic agonist.

4. The method of clause 3, wherein the β₂-adrenergic agonist is selected from the group consisting of formoterol, albuterol, isoproterenol, pirbuterol, levalbuterol, clenbuterol, salmeterol, indacaterol, and vilanterol.

5. The method of clause 1, wherein the adenylyl cyclase activator is forskolin or colforsin.

6. The method of any of the preceding clauses, wherein the cholinergic agonist is a direct acting cholinergic agonist.

7. The method of clause 6, wherein the direct acting cholinergic agonist is selected from the group consisting of methacholine, acetylcholine, bethanechol, pilocarpine, and carbachol.

8. The method of any of the preceding clauses, wherein the β-adrenergic agonist and the cholinergic agonist are administered sequentially.

9. The method of clause 8, wherein the β-adrenergic agonist is administered prior to the cholinergic agonist.

10. The method of any of clauses 1-9, wherein the β-adrenergic agonist and the cholinergic agonist are administered simultaneously.

11. The method of any of the preceding clauses, wherein the β-adrenergic agonist and the cholinergic agonist are administered systemically.

12. The method of any of clauses 1-10, wherein the β-adrenergic agonist and the cholinergic agonist are administered locally.

13. The method of any of the preceding clauses, wherein the administration does not cause airway smooth muscle contractions.

14. The method of any of the preceding clauses, further comprising administering one or more cystic fibrosis transmembrane conductance regulator (CFTR) modulators.

15. The method of clause 14, wherein the one or more CFTR modulators are Elexacaftor, Tezacaftor, Deutivacaftor, Vanzacaftor, and Ivacaftor.

16. The method of any of the preceding clauses, wherein the administration results in a synergistic increase in mucus transport relative to the mucus transport of either agonist used alone.

17. The method of any of the preceding clauses, wherein the individual is a human.

18. A method of increasing the rate of airway submucosal gland secretion in an individual, the method comprising:
administering to the individual a β-adrenergic agonist or an adenylyl cyclase activator, in combination with a cholinergic agonist to increase the rate of airway submucosal glad secretion in the individual.

19. The method of clause 18, wherein the-adrenergic agonist is a β₂-adrenergic agonist.

20. The method of clause 19, wherein the β₂-adrenergic agonist is selected from the group of formoterol, albuterol, isoproterenol, pirbuterol, levalbuterol, clenbuterol, salmeterol, indacaterol, and vilanterol.

21. The method of clause 18, wherein the adenylyl cyclase activator is forskolin or colforsin.

22. The method of any of clauses 18-21, wherein the cholinergic agonist is a direct acting cholinergic agonist.

23. The method of clause 22, wherein the direct acting cholinergic agonist is selected from the group consisting of methacholine, acetylcholine, bethanechol, pilocarpine, and carbachol.

24. The method of any of clauses 18-23, wherein the β-adrenergic agonist and the cholinergic agonist are administered sequentially.

25. The method of any of clauses 18-23, wherein the β-adrenergic agonist and the cholinergic agonist are administered simultaneously.

26. The method of any of clauses 18-25, wherein the β-adrenergic agonist and the cholinergic agonist are administered systemically.

27. The method of any of clauses 18-26, wherein the administration results in a synergistic increase in submucosal glad secretion rate relative to the submucosal glad secretion rate of either agonist used alone.

28. The method of any of clauses 18-27, wherein the individual is a mammal.

29. The method of any of clauses 18-27, wherein the individual is a pig.

30. The method of any of clauses 18-27, wherein the individual is a ferret.

31. The method of any of clauses 18-27, wherein the individual is a human.

32. The method of any of clauses 18-31, wherein the individual has a mutation in the cystic fibrosis transmembrane conductance regulator (CFTR) gene resulting in a non-functional CTFR protein, a CTFR protein with reduced functionality, or no CFTR protein.

33. A method of suppressing cholinergic agonist induced airway smooth muscle contraction in an individual, the method comprising:
administering to the individual a β-adrenergic agonist or an adenylyl cyclase activator, wherein the administration of the β-adrenergic agonist or adenylyl cyclase activator occurs before or at the same time that the individual has been administered a cholinergic agonist to suppress the airway smooth muscle contraction.

34. The method of clause 33, wherein the β-adrenergic agonist is a β₂-adrenergic agonist.

35. The method of clause 34, wherein the β₂-adrenergic agonist is selected from the group of formoterol, albuterol, isoproterenol, pirbuterol, levalbuterol, clenbuterol, salmeterol, indacaterol, and vilanterol.

36. The method of clause 35, wherein the adenylyl cyclase activator is forskolin or colforsin.

37. The method of any of clauses 34-36, wherein the cholinergic agonist is a direct acting cholinergic agonist.

38. The method of clause 37, wherein the direct acting cholinergic agonist is selected from the group consisting of methacholine, acetylcholine, bethanechol, pilocarpine, and carbachol.

39. The method of any of clauses 34-38, wherein the β-adrenergic agonist is administered systemically.

40. The method of any of clauses 34-38, wherein the β-adrenergic agonist is administered locally.

41. The method of any of clauses 34-40, wherein the individual is a mammal.

42. The method of any of clauses 34-40, wherein the individual is a pig.

43. The method of any of clauses 34-40, wherein the individual is a ferret.

44. The method of any of clauses 34-40, wherein the individual is a human.

45. The method of any of clauses 34-44, wherein the individual has a mutation in the cystic fibrosis transmembrane conductance regulator (CFTR) gene resulting in a non-functional CTFR protein or a CTFR protein with reduced functionality.

46. A pharmaceutical composition comprising:
a β-adrenergic agonist or an adenylyl cyclase activator;
a cholinergic agonist; and
a pharmaceutical excipient.

47. The method of clause 46, wherein the β-adrenergic agonist is a β₂-adrenergic agonist.

48. The method of clause 47, wherein the β₂-adrenergic agonist is selected from the group of formoterol, albuterol, isoproterenol, pirbuterol, levalbuterol, clenbuterol, salmeterol, indacaterol, and vilanterol.

49. The method of clause 46, wherein the adenylyl cyclase activator is forskolin or colforsin.

50. The method of any of clauses 46-49, wherein the cholinergic agonist is a direct acting cholinergic agonist.

51. The method of clause 50, wherein the direct acting cholinergic agonist is selected from the group consisting of methacholine, acetylcholine, bethanechol, pilocarpine, and carbachol.

52. The composition of any of clauses 46-51, wherein the compositions is formulated for systemic administration.

53. The composition of any of clauses 46-52, wherein the compositions is formulated for local administration.

54. The composition of any of clauses 46-53, wherein the composition is a solid composition.

55. The composition of any of clauses 46-53, wherein the composition is a liquid composition.

56. A device, the device comprising the composition of any of clauses 46-55.

57. The device of clause 56, wherein the device is an oral inhaler.

58. The device of clause 56, wherein the device is a nasal inhaler.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating an individual for a muco-obstructive disorder, the method comprising:
administering to the individual a β-adrenergic agonist or an adenylyl cyclase activator, in combination with a cholinergic agonist to treat the individual for the muco-obstructive disorder.

2. The method of claim 1, wherein the muco-obstructive disorder is selected from the group consisting of: cystic fibrosis, primary ciliary dyskinesia, asthma, chronic obstructive pulmonary disease (COPD), idiopathic pulmonary fibrosis, chronic bronchitis, and non-CF bronchiectasis.

3. The method of claim 1, wherein the β-adrenergic agonist is a β₂-adrenergic agonist.

4. The method of claim 3, wherein the β₂-adrenergic agonist is selected from the group consisting of formoterol, albuterol, isoproterenol, pirbuterol, levalbuterol, clenbuterol, salmeterol, indacaterol, and vilanterol.

5. The method of claim 1, wherein the adenylyl cyclase activator is forskolin or colforsin.

6. The method of any of the preceding claims, wherein the cholinergic agonist is a direct acting cholinergic agonist.

7. The method of claim 6, wherein the direct acting cholinergic agonist is selected from the group consisting of methacholine, acetylcholine, bethanechol, pilocarpine, and carbachol.

8. The method of any of the preceding claims, wherein the β-adrenergic agonist and the cholinergic agonist are administered sequentially.

9. The method of claim 8, wherein the β-adrenergic agonist is administered prior to the cholinergic agonist.

10. The method of any of claims 1-9, wherein the β-adrenergic agonist and the cholinergic agonist are administered simultaneously.

11. The method of any of the preceding claims, wherein the β-adrenergic agonist and the cholinergic agonist are administered systemically.

12. The method of any of claims 1-10, wherein the-adrenergic agonist and the cholinergic agonist are administered locally.

13. The method of any of the preceding claims, wherein the administration does not cause airway smooth muscle contractions.

14. The method of any of the preceding claims, further comprising administering one or more cystic fibrosis transmembrane conductance regulator (CFTR) modulators.

15. The method of claim 14, wherein the one or more CFTR modulators are Elexacaftor, Tezacaftor, and Ivacaftor.

16. The method of any of the preceding claims, wherein the administration results in a synergistic increase in mucus transport relative to the mucus transport of either agonist used alone.

17. The method of any of the preceding claims, wherein the individual is a human.

18. A pharmaceutical composition comprising:
a β-adrenergic agonist or an adenylyl cyclase activator;
a cholinergic agonist; and
a pharmaceutical excipient.

19. The composition of claim 18, wherein the β-adrenergic agonist or adenylyl cyclase activator is selected from the group of formoterol, albuterol, isoproterenol, pirbuterol, levalbuterol, clenbuterol, salmeterol, indacaterol, vilanterol, forskolin, and colforsin.

20. The composition of claim 19, wherein the direct acting cholinergic agonist is selected from the group consisting of methacholine, acetylcholine, bethanechol, pilocarpine, and carbachol.
